(19) United States
(12) Patent Application Publication
(10) Pub. No.: US 2026/0242744 A1
(43) Pub. Date: Aug. 20, 2026

(54) CONSTRUCTS FOR REPROGRAMMING OF SMOOTH MUSCLE CELLS INTO ENDOTHELIAL CELLS

(71) Applicant: The Brigham and Women's Hospital, Inc., Boston, MA (US)

(72) Inventor: Mark W. Feinberg, Newton, MA (US)

(21) Appl. No.: 19/159,829

(22) PCT Filed: Feb. 27, 2024
(86) PCT No.: PCT/US2024/017541

§ 371 Date: Aug. 26, 2025

Related U.S. Application Data
(60) Provisional application No. 63/449,022, filed on Feb. 28, 2023.

(51) Int. Cl. C12N 5/071; A61K 35/44; A61P 9/10; C12N 15/86

(52) U.S. Cl. CPC C12N 5/069; A61K 35/44; A61P 9/10; C12N 15/86; C12N 2501/65; C12N 2506/1338; C12N 2510/00; C12N 2750/14143; C12N 2830/008; C12N 2830/48

(57) ABSTRACT

Described herein are compositions and methods using miR-143-3p and/or miR-145-5p inhibitors coupled with miR-146a-5p and miR-181b-5p mimics, to promote the transdifferentiation of vascular smooth muscle cells or fibroblasts into endothelial cells, and to treat certain cardiovascular conditions.

### CLAIM OF PRIORITY

This application claims the benefit of U.S. Provisional Patent Application Ser. No. 63/449,022, filed on Feb. 28, 2023. The entire contents of the foregoing are hereby incorporated by reference.

### TECHNICAL FIELD

Described herein are compositions and methods using miR-143-3p and/or miR-145-5p inhibitors coupled with miR-146a-5p and miR-181b-5p mimics, to promote the transdifferentiation of vascular smooth muscle cells or fibroblasts into endothelial cells, and to treat certain cardiovascular conditions.

### BACKGROUND

The innermost lining of the blood vessel wall comprise of a monolayer of endothelial cells (ECs), which function as a barrier between the blood and tissues (1). Damage to the vasculature inner wall from tissue trauma or mechanical intervention such as balloon angioplasty or stent placement can result in denuding of the endothelium, leading to diminished vessel utility (2). These sites of endothelial damage are prone to rapid atherosclerotic events, vessel delamination, and restenosis due to neointima formation as a result of proliferation of medial smooth muscle cells (SMCs) and adventitia fibroblasts (3). However, regeneration of the endothelium is a notoriously sluggish process as EC growth is relatively slow when compared to perivascular cells such as SMCs. Many therapies have attempted to use progenitor cells or induced pluripotent stem cells in order to facilitate repair and reendothelialization of the vessel (4, 5). However, optimizing these therapies and techniques requires spatial and cellular resolution that is frequently obviated by cell source and individual pathologies. Recent advances in transcriptome profiling and cell lineage conversion techniques have broadened possible therapeutic routes as perivascular host cells may be a possible regenerative source (6). Of these techniques, cellular reprograming through targeting microRNAs (miRNAs) is of particular interest due to their profound regulatory effects in proliferation, differentiation, and function (7, 8).

### SUMMARY

Described herein are constructs for expression of miRNAs and inhibitors that can be used to transdifferentiate vascular smooth muscle cells or fibroblasts into endothelial cells. As demonstrated herein, miR-143-3p and/or miR-145-5p inhibitors coupled with miR-146a-5p and miR-181b-5p mimics were sufficient for accomplishing this transformation. This transdifferentiation protocol can be used to generate inducible endothelial cells (iECs) that are transcriptionally, phenotypically, and functionally similar to other endothelial cells. This miRNA-engineered approach is useful in a range of cardiovascular-based therapies.

Provided herein are methods for generating a population of inducible endothelial cells (iECs). The methods comprise (a) providing a population of cells comprising smooth muscle cells (SMCs) or fibroblasts; (b) contacting the population of cells comprising SMCs or fibroblasts with an expression construct for expression of transdifferentiation factors, wherein the expression construct comprises one or both of (i) one, two, or more tough decoy RNA expression sequences for inhibiting miR-143 and miR-145 that is at least 80%, 85%, 90%, 95%, or 99% identical to SEQ ID NO:48, and/or (ii) a miR-146a/miR-181b-2 miRNA mimic expression sequence that is at least 80%, 85%, 90%, 95%, or 99% identical to SEQ ID NO:43, in an amount and for a time sufficient to induce transdifferentiation of the SMCs or fibroblasts to iECs (though transdifferentiation may take longer), e.g., for at least 2, 4, 6, 8, 10, 12, 14, 16, 18, 24, 36, or 48 hours, up to 48 or 73 hours or longer; and (c) culturing the cells in supportive media, preferably for at least 10, 12, 14, 16, 18, or 20 days, thereby generating a population of iECs.

In some embodiments, the SMC are isolated from aorta (AoSMC), coronary artery (CASMC), pulmonary artery (PASMC), umbilical artery (UASMC), bladder smooth muscle cells (HBdSMC), or derived from adipose tissue smooth muscle cells or progenitors (ASMCs), or blood derived circulating smooth muscle cell progenitors (SPCs). In some embodiments, the fibroblasts are from skin (dermal fibroblasts), lung fibroblasts, cardiac fibroblasts, aortic fibroblasts, adipose tissue fibroblasts, or foreskin fibroblasts.

In some embodiments, the SMCs or fibroblasts are contacted in vitro or in vivo.

In some embodiments, the expression construct is administered by injection for endothelial repair.

In some embodiments, the expression construct is administered in a composition comprising liposomes, optionally wherein the liposomes encapsulate expression construct.

In some embodiments, the expression construct is administered as DNA or RNA sequences encoding the transdifferentiation factors, optionally in a plasmid or viral vector. In some embodiments, the viral vector is a retrovirus, adenovirus, adeno-associated virus (AAV), or lentivirus. In some embodiments, the AAV is an AAV9, AAV2, or AAV2.5. In some embodiments, the expression construct comprises a promoter that drives expression in smooth muscle cells, e.g., SM22a promoter sequence to drive expression of miRNA.

Also provided herein are compositions comprising an expression construct encoding transdifferentiation factors comprising one or both of (i) a tough decoy expression sequence for inhibiting miR-143 and miR-145 that is at least 80%, 85%, 90%, 95%, or 99% identical to SEQ ID NO:48, and/or (ii) a miR-146a/miR-181b-2 miRNA mimic expression sequence that is at least 80%, 85%, 90%, 95%, or 99% identical to SEQ ID NO:43, optionally in a carrier, optionally a pharmaceutically acceptable carrier. In some embodiments, the expression construct comprises two individual RNAs or DNAs, or a single RNA or DNA. In some embodiments, the compositions comprise a single expression construct comprising (i) a tough decoy expression sequence for inhibiting miR-143 and miR-145 that is at least 80%, 85%, 90%, 95%, or 99% identical to SEQ ID NO:48, and (ii) a miR-146a/miR-181b-2 miRNA mimic expression sequence that is at least 80%, 85%, 90%, 95%, or 99% identical to SEQ ID NO:43.

In some embodiments, the composition comprises liposomes, optionally wherein the liposomes encapsulate the expression construct.

In some embodiments, the expression construct is present in the composition as DNA sequences encoding the transdifferentiation factors, optionally in a plasmid or viral vector. In some embodiments, the viral vector is a retrovirus, adenovirus, adeno-associated virus (AAV), or lentivirus. In some embodiments, the AAV is an AAV9, AAV2, or AAV2.5. In some embodiments, the expression construct comprises a promoter that drives expression in smooth muscle cells, e.g., SM22a promoter sequence, and/or optionally a woodchuck hepatitis virus post-transcriptional regulatory element (WPRE).

Additionally, provided herein are methods of treating a subject, and the compositions described herein for use in a method of treating a subject, the method comprising administering to the subject a therapeutically effective amount of: (i) a population of inducible endothelial cells (iECs) generated by a method described herein; or (ii) a composition as described herein.

In some embodiments, the subject has or is at risk of developing endothelial injury. In some embodiments, the subject has or is at risk of developing endothelial injury as a result of a planned or past cardiovascular intervention. In some embodiments, the cardiovascular interventions comprises angioplasty, stent placement, catheter ablation, heart valve surgery, or bypass surgery.

In some embodiments, the subject has a disorder associated with ischemic injury. In some embodiments, the disorder associated with ischemic injury is myocardial infarction, ischemic stroke, ischemic renal injury, limb ischemia, arteriovenous (AV) fistula injury, organ transplant graft injury, or a wound healing.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-F: miRNAs can efficiently reprogram smooth muscle cells to endothelial cells. (A) Schematic for direct reprogramming of human coronary artery smooth muscle cells (CASMCs) to generate inducible endothelial cells (iECs) by defined miRNAs. Briefly, miR-143-3p, miR-145-5p, miR-146a-5p and miR-181b-5p were identified as potential targets due to differential expression in smooth muscle cells and endothelial cells. CASMCs were transfected and expanded in endothelial growth media prior to cell sorting for endothelial cell markers. Sorted iECs were used in subsequent analyses. (B) Representative phase contrast and f-actin-stained confocal micrographs of CASMCs, iECs, and human umbilical vein endothelial cells (HUVECs). Scale bars are 500 mm. C) RT-qPCR results of selected endothelial gene markers: VE-Cadherin, KDR, SELE, vWF, and CD31 and smooth muscle gene markers: Calponin, ACTA2, and MYH1l. All samples were normalized to GAPDH. (D)-(F) Protein abundance and quantification of selected endothelial cell markers: VCAM1, SELE, vWF, and VE-cadherin and smooth muscle cell markers: MYH11, Vimentin, αSMA, and Calponin were detect by Western blot analysis. GAPDH was used as a loading control. All quantifications are representative of n=4 biological replicates. All values are mean+/−SEM. P-values are indicated by *, **, ***, and **** and indicate a value of <0.05, <0.01, <0.001, and <0.0001, respectively. Data was analyzed using a one-way ANOVA with Tukey's post-hoc test.

FIGS. 2A-F: iECs exhibit increased endothelial cell protein expression and internalize Ac-LDL and oxLDL. (A) and (B) Confocal micrographs and respective quantification of endothelial cell markers of confluent monolayers for CASMCs, iECs, and HUVECs. DAPI was used as a counter stain for nuclei visualization. Scale bars are 50 μm. Quantification was normalized to fluorescence intensities in HUVECs. (C) and (D) Confocal micrographs and respective quantification of smooth muscle cell markers of confluent monolayers for CASMCs, iECs, and HUVECs. DAPI was used as a counter stain for nuclei visualization. Scale bars are 50 μm. Quantification was normalized to fluorescence intensities in CASMCs. (E) Confocal micrographs of DiI-labeled acetylated LDL (Ac-LDL) or oxidized LDL (oxLDL) uptake in CASMCs, iECs, and HUVECs. (F) Percent positive cell quantification of DiI-labeled Ac-LDL or oxLDL uptake in CASMCs, iECs, and HUVECs. All quantifications are representative of n=4 biological replicates and 3 averaged fields of view per sample. All values are mean+/−SEM. P-values are indicated by *, **, and **** and indicate a value of <0.05, <0.01, and <0.0001, respectively. Data was analyzed using a one-way ANOVA with Tukey's post-hoc test.

FIGS. 3A-D: RNA-sequencing and genome-wide profiling of CASMCs, iECs, and HUVECs reveal an endothelial-like transcriptome for iECs. (A) Principal component analysis of the whole transcriptome of CASMCs, iECs, and HUVECs. n=4 biological replicates for sequencing. (B) Individual associations between each RNA-seq sample and their comparative principal component Euclidian distance. (C) Unsupervised hierarchical heatmap cluster analysis of each individual sample. (D) Heatmap of CASMC, iEC, and HUVEC samples that have been organized by selected endothelial and smooth muscle cell genes.

FIGS. 4A-C: Gene set enrichment analysis indicate overlap in upregulated endothelial pathways in iECs and HUVECs. (A) Venn diagram representing selected differentially expressed genes (Log₂(FC)>2.0, p_{adj}<0.05) across each group.

Intersections indicate overlapping genes. (B) Gene set enrichment analysis using MetaCore process networks of common differentially expressed genes in iECs and HUVECs. Process networks are displayed as a dot plot and sorted in descending order (−Log₁₀(FDR)). Dot size represents the number of genes changed in the process network and dot color represents the fraction of differentially expressed genes within the network. (C) Chord plot representing the differentially regulated genes for the four top process networks: “Blood Vessel Morphogenesis”, “Neurogenesis”, “Regulation of Angiogenesis”, and “Platelet-Endothelium-Leukocyte Interactions”.

FIGS. 5A-C: Transcriptomic differences between iECs and CASMCs reveal downregulation of many smooth muscle cell pathways. (A) Volcano plot displaying differentially expressed genes (Log₂(FC)>2.0, p_{adj}<0.05) between iECs and CASMCs with selected significant genes enumerated. (B) Log₂(FC) distribution and gene ranking of iEC and CASMC comparisons, with endothelial and smooth muscle cell enriched genes highlighted. (C) Gene set enrichment analysis using MetaCore process networks of differentially expressed genes in iECs and CASMCs, sorted by −Log₁₀(FDR). Dot size represents the number of genes changed in the process network and dot color represents the fraction of differentially expressed genes within the network.

FIGS. 6A-G: iECs exhibit endothelial cell functionality and VEGF responsivity. (A) Time-lapse, brightfield images of endothelial tube formation on Matrigel™ over 16 hours in HUVECs, iECs, and CASMCs. (B) Quantification of tube number in HUVECs, iECs, and CASMCs in 4-hour increments. n=4 biological replicates. (C) DAPI-stained underside of transwell inserts coated with CASMCs, iECs and HUVECs, with and without 50 ng/mL of VEGF for 12 hours and (D) the quantification of number of migrated cells indicated by number of nuclei. Scale bars are 100 μm. n=4 biological replicates and averaged 3 fields of view per transwell. (E) Protein abundance of eNOS and p-eNOS in CASMC, iEC and HUVECs in response to VEGF stimulation for 0, 1, 10, and 30 minutes. (F) Quantification of relative p-eNOS signal, normalized to eNOS. n=3 Western blots. (G) Quantification of BrdU microplate assay of CASMCs, iECs, and HUVECs in response to 10, 50, and 100 ng/mL of VEGF after 24 hours. n=6 technical replicates. All values are mean+/−SEM. P-values are indicated by *, **, ***, and **** and indicate a value of <0.05, <0.01, <0.001, and <0.0001, respectively. Data was analyzed using a one-way ANOVA with Tukey's post-hoc test.

FIGS. 7A-E: iECs enhance blood flow recovery after hindlimb ischemia. (A) Representative Doppler images of the right and left paws following hindlimb ischemia in the left paw at 0, 4, 7, and 11 days for mice injected with PBS, HUVECs, and iECs in the tail vein. (B) Quantification of Doppler images indicating blood flow recovery. Recovery percentage compares the injured limb to the contralateral limb. n=8 for PBS, 8 for HUVECs, and 10 for iECs. (C) Representative photographs of mouse limbs for visual assessment of hindlimb damage and recovery at 0, 4, 7, and 11 days in mice. (D, E) Quantification of nail and toe/limb loss in mice. n=9 for PBS, 10 for HUVECs, and 10 for iECs. All values are mean+/−SEM. P-values are indicated by *, ***, and **** and indicate a value of <0.05, <0.001, and <0.0001, respectively. Data was analyzed using a mixed two-way ANOVA with Tukey's post-hoc test.

FIGS. 8A-E: Discovery of a combination of miRNAs that reprograms CASMCs to iECs. (A) Microarray data detailing the miRNA transcription expression profile differences between human arterial SMCs and ECs. (B) Raw transcript counts of microarray profiling. n=6 for CAECs and n=4 for HAoSMCs. (C) RT-qPCR of miRNA combination demonstrating similar expression levels between CASMCs and HAoSMCs. (D, E) Relative protein expression of endothelial cell and smooth muscle cell markers in CASMCs following transfection of single and combination of miRNAs identified in microarray profiling. Samples were normalized to beta actin.

FIGS. 9A-B: Expression of EC miRNAs in healthy and diseased ECs. (A) Expression of miR-146a-5p and miR-181-5p in endothelial cells from heart, gastrocnemius muscle, and aorta in 12-week-old Db/− and Db/Db mice. N=3 biological replicates. (B) Expression of miR-146a-5p and miR-181b-5p in HAECs derived from non-diabetic (HAEC) and diabetic donors (T2DM HAEC). All values are mean+/−SEM. P-values are indicated by *, ***, and **** and indicate a value of <0.05, <0.001, and <0.0001, respectively. Data was analyzed using a one-way ANOVA with Tukey's post-hoc test.

FIG. 10: CAECs and iEC express similar EC markers. Protein abundance and quantification of selected endothelial cell markers: CD31, VE-Cadherin, and KDR were detected by Western blot analysis. GAPDH was used as a loading control.

FIGS. 11A-D: Isolation and generation of miECs from aortic fibroblasts. (A) Schematic for direct reprogramming of mouse aortic fibroblasts to generate iECs (miECs) by transfection with miR-143-3p, miR-145-5p, miR-146a-5p, and miR-181b-5p. Cells were isolated by antibody-labeled magnetic beads for CD90.2 and transfected utilizing the same iEC protocol. (B) Protein abundance and quantification of CD31 and VE-Cadherin were detected by Western blot analysis. GAPDH was used as a loading control. (C) RT-qPCR results for CD31 and VE-Cadherin. All samples were normalized to GAPDH. N=3 technical replicates. (D) Confocal micrographs of mouse fibroblasts (mFibroblasts), miECs, and HUVECs stained against CD31 and VE-Cadherin. DAPI was used as a counter stain for nuclei visualization. Scale bars are 50 μm. All values are mean+/−SEM. P-values are indicated by *, **, and **** and indicate a value of <0.05, <0.01, and <0.0001, respectively. Data was analyzed using a one-way ANOVA with Tukey's post-hoc test.

FIGS. 12A-C: Differentially expressed transcripts in iECs and HUVECs. (A) Venn diagram and volcano plot displaying differentially expressed genes (Log 2(FC) and padj<0.05) between iECs and HUVECs. (B) Heatmap of top 10 increased and reduced gene expressions for iECs when compared to HUVECs. (C) Gene set enrichment analysis using MetaCore process networks of differentially expressed genes in iECs and HUVECs. Process networks are displayed as a dot plot and sorted in descending order (−Log 10(FDR)). Dot size represents the number of genes changed in the process network and dot color represents the fraction of differentially expressed genes within the network.

FIGS. 13A-C: Top differentially expressed transcripts in CASMCs and iECs. (A) Heatmap of top 10 increased and reduced gene expressions for iECs when compared to CASMCs. (B) Detailed gene network visualization of “Blood Vessel Morphogenesis” demonstrating the relationship between endothelial enriched genes and cell adhesion molecules, VEGF receptors, and Notch signaling. (C) Detailed gene network visualization of “Cell-Matrix Interactions” demonstrating the relationship between smooth muscle enriched genes such collagen synthesis and metalloproteinases.

FIGS. 14A-B: iECs may be generated using a 3-miRNA combination. CASMCs were transfected with miR-146a-5p, miR-181-5p, and anti-miR-145-5p using the same iEC transdifferentiation protocol. (A) Protein abundance of VE-Cadherin, CD31, and KDR were detect by Western blot analysis. GAPDH was used as a loading control. (B) RT-qPCR results for VE-Cadherin, CD31, and KDR. All samples were normalized to GAPDH. N=3 technical replicates. All values are mean+/−SEM. P-values are indicated by *, **, ***, and **** and indicate a value of <0.05, <0.01, <0.001, and <0.0001, respectively. Data was analyzed using a one-way ANOVA with Tukey's post-hoc test.

FIG. 15: AAV pSM22a-GFP control was added at the indicated MOI to human coronary artery smooth cells (CASMCs) or the mouse SMC cell line MOVAS. Immunofluorescence imaging for GFP showed successful transduction of both CASMCs and MOVAS.

FIGS. 16A-B: AAV pSM22a-GFP control or AAV pSM22a-miRNA cassette-GFP was added at 10⁴ MOI to the mouse SMC cell line MOVAS (16A) or human coronary artery smooth cells (CASMCs)(16B). Cells were FACS sorted for GFP after 7 days and subjected to RTqPCR for miR-143, miR-145, miR-146a, and miR-181b. The data show that AAV pSM22a-miRNA cassette-GFP successfully inhibited the expression for miR-143 and miR-145 and overexpressed miR-146a and miR-181b in both cell lines.

FIGS. 17A-D: AAV pSM22a-GFP control or AAV pSM22a-miRNA cassette-GFP was added at 10⁴ MOI to the mouse SMC cell line MOVAS (17A-B) or human coronary artery smooth cells (CASMCs)(17C-D). Cells were FACS sorted for GFP after 7 days and subjected to RTqPCR for endothelial markers CD31, KDR, and VE-cadherin and vascular smooth muscle cell markers ACTA2, vimentin, TGLN/CNN1, or Myh11 as indicated. The data show that the AAV pSM22a-miRNA cassette-GFP successfully increased endothelial markers and repressed vascular smooth muscle cell markers.

### DETAILED DESCRIPTION

Micro RNAs (miRNAs) are short, single-stranded endogenous noncoding RNAs with seed sequences that target regions of mRNAs to translationally repress or for degradation (9). Interestingly, numerous miRNAs expression profiles are tied to specific tissues and cell populations, indicating that they are necessary for cell microenvironment specificity (10). Endothelial miRNAs have been identified to play key roles in angiogenic processes and are frequently dysregulated in vasculoproliferative diseases such as atherosclerosis and tumor angiogenesis (11, 12). Similarly, vascular SMCs express unique miRNA transcripts such as miR-143 and miR-145 that contribute to their overall functionality as a result of their interactions with cell processes involving contractility or proliferation (13). In vasculoproliferative disease states, an increase in cell plasticity and dysfunction such as SMC proliferation is associated with changes to the miRNA transcriptome (14). Several studies have identified a miRNA communication between ECs and SMCs whereby each is modulating the others behavior in response to physiological changes (15, 16). Indeed, nascent observations demonstrate ECs undergo transformation into SMCs as a result of direct and indirect miRNA reprogramming (17). However, few studies have reported or attempted the process of driving SMCs into ECs through direct miRNA reprogramming. The abundancy and proximity of vascular SMCs to the intima would make them an ideal pool source for regenerating ECs in damaged vessel walls if they were capable of transdifferentiation.

Regulatory miRNA networks are known to play a role in multiple aspects of vascular biology and are coupled to context-driven dependencies that are endemic to smooth muscle and endothelial cell function (7, 27). While the molecular underpinnings of many of these miRNAs have been characterized to some degree, interference in miRNA signaling as a means of generating a transdifferentiated endothelial cell population has been insufficiently investigated. Herein, we developed a novel protocol, based upon a hypothesis-generating miRNA microarray profiling, capable of transforming a population of smooth muscle cells into endothelial-like cells. This protocol follows a transdifferentiation procedure of 10-45 days or more, e.g., 12-20 or 12-45 days, generally comprising (a) contacting smooth muscle cells (SMCs, e.g., human SMCs) or fibroblasts with an expression construct that comprises one or both of (i) one, two, or more tough decoy RNA expression sequences for inhibiting miR-143 and miR-145 that is at least 80%, 85%, 90%, 95%, or 99% identical to SEQ ID NO:48, and/or (ii) a miR-146a/miR-181b-2 miRNA mimic expression sequence that is at least 80%, 85%, 90%, 95%, or 99% identical to SEQ ID NO:43, (b) growth of transfected cells in supportive media; and (c) sorting for ICAM-1 followed by expansion of transfected cells. This approach was subsequently validated in our analyses as iECs were found to be transcriptionally, phenotypically, and functionally similar to ECs. Moreover, this approach utilized adult mature SMCs or fibroblasts as opposed to immature cell-types such as the commonly used fibroblasts from human foreskin or other progenitor subsets. The use of this miRNA combination is consistent with some of the known properties for each of the miRNAs implicated in smooth muscle cell and endothelial cell function and dysfunction (28′-30). As used herein, the term “cassette” means a combination of miRNAs, and does not require the miRNAs to be present in a single nucleic acid (though they may be).

RNA-seq analysis of iECs revealed similar transcriptional profiling with ECs. Indeed, quantitative index showed a greater similarity between iECs and HUVECs than with CASMCs with respect to their global transcriptome, showing enrichment on blood vessel morphogenesis, endothelium-leukocyte interaction, and angiogenesis pathways. Detailed gene network visualization (FIGS. 13A-C) highlighted specific genes related to endothelial functions such as vasoactive mediators (eNOS, EDN1), leukocyte interaction through cell adhesion molecules (VCAM1, ICAM1, SELE), and VEGF signaling (FLTI, KDR, FLT4), In this regard, previous studies on transdifferentiated EC from fibroblasts showed cell cycle-related pathways in addition to angiogenesis among the most significative enriched pathways, while genes involved in extracellular matrix pathways w ere downregulated in transdifferentiated ECs compared to fibroblasts (6). Similar results were observed in our iECs with enrichment analysis showing downregulation of genes related to connective tissue degradation, cell-matrix interactions, and extracellular matrix remodeling in iECs compared to CASMCs. In a recent study, Hong et al. converted SMC towards endothelial-like cells by dedifferentiation to a vascular progenitor state, introducing OCT4, SOX2, KLF4 and c-YC transcription factors, and then used endothelial culture conditions (26). Transcriptome-wide analysis on these SMC-derived ECs showed upregulation of endothelial enriched genes, while smooth muscle genes were downregulated. In addition, RNA-Seq analysis highlighted the upregulation of Notch pathway genes in SMC-derived ECs. Further analysis using gain and loss-of-function revealed JAG1 as an important regulator of SMC-derived ECs generation. Accordingly, our transcriptomic data showed Notch signaling among the top iECs enriched pathways, which includes several related genes such as NOTCH1, DLL4. and JAG1. Therefore, RNA-seq data validated the endothelial-like phenotype at the transcriptional level, and highlighted key genes and pathways involved in the generation of iECs mediated by the 4-miRNA combination.

In addition, iEC transcriptomic data along with gene enrichment analysis of predicted targets from the transfected miRNA mimics and inhibitors highlighted other pathways, including Wnt signaling, Hedgehog signaling, PI3K/AKT signaling, MAPK signaling, JAK/STAT signaling, or Integrin signaling, among others, that may provide relevant signaling pathways in endothelial transdifferentiation, Indeed, using publicly available RNA-seg datasets (GSE116464) to compare the transcriptome of our iECs to human induced pluripotent stem cell-derived ECs (iPSC-ECs) and human primary fetal endothelial cells (hF-ECs), there was greater similarity among iECs and HUVECs, hF-ECs, and iPSC-ECs compared to CASMCs using both PCA plots and hierarchical heatmap clustering (51).

Previous miRNA investigations have demonstrated the capability of sets of miRNAs to transdifferentiate lineage-committed cells. For example, miR-1, miR-133, miR-208, and miR-499 mimics were capable of coordinative reprogramming of cardiac fibroblasts into cardiomyocytes in vitro and in vivo (52, 53). The introduction of cardiac transcription factors such as GATA4. Hand2, myocardin, and others have been used in combination with these miRNAs in order to enhance efficiency (54). Individual and families of miRNAs have also been identified that appear to be involved in the differentiation of ECs from progenitors and potentially robust targets may be of interest in future studies. (55, 56). Similarly, injections of endothelial progenitor cells transfected with miR-326-5p into mice after surgical induction of myocardial infarction found a decrease in cardiac fibrosis and increase in arteriole density, highlighting the therapeutic capacity of cells primed with pro-angiogenic miRNAs.

The present observations that iECs perform better in blood flow recovery experiments may be due to their induced plasticity rather than the lineage commitment of a differentiated EC such as HUVECs. While we could not detect engraftment of either HUVEC- or iEC-injected mice at the end of the hindlimb ischemia study, this may reflect several possibilities: (1) the survival of these cells at this later time point may be limited; (2) the main contribution to neovascularization is likely a paracrine effect that is more prominent early after ischemic injury; or (3) there may be technical limitations with immunofluorescent staining, whereas cell labeling prior to injection might allow for improved sensitivity.

Described herein are methods and compositions for regenerating endothelial populations from SMCs, e.g., pre-existing mural cells, or fibroblasts, overcoming previous hurdles requiring the recruitment or differentiation of progenitor cell types. In some embodiments, this is accomplished with the transdifferentiation of SMCs or fibroblasts into iECs using transdifferentiation factors as described herein, e.g., a 4-miRNA cassette (combination of miRNAs), followed by enrichment in supportive growth media. The iECs were found to be transcriptionally, phenotypically, and functionally similar to ECs. The present methods can be used in therapeutic applications, including in subjects undergoing a cardiovascular intervention, as endothelial denudation followed by neointimal proliferation and restenosis is a frequent occurrence during cardiovascular intervention. These cells also serve as a scalable source of ECs for disease states associated with ischemic injury due to their pro-angiogenic functions. Recruitment and transdifferentiation of host perivascular SMCs or fibroblasts into ECs provides a regenerative approach for restoring endothelial homeostasis and improving clinical outcomes.

Transdifferentiation Factors for Conversion of SMCs or Fibroblasts to iECs

The methods comprise contacting or expressing in the smooth muscle cells or fibroblasts transdifferentiation factors comprising (i) miR-143-3p and/or miR-145-5p inhibitors and (ii) 146a-5p and 181b-5p mimics. Table 1 provides the sequences of each of these miRNAs.

TABLE 1
miRNA Transdifferentiation Factors

| | miRBase | | | SEQ |
|---|---|---|---|---|
| Stem- | Stem-loop | Mature | | ID |
| loop ID | Acc. No. | miRNA ID | Mature miRNA Sequence | NO: |
| hsa- | MI0000459 | hsa-miR-143- | UGAGAUGAAGCACUGUAGCUC | 1 |
| miR-143 | | 3p | | |
| hsa- | MI0000461 | hsa-miR-145- | GUCCAGUUUUCCCAGGAAUCCCU | 2 |
| miR-145 | | 5p | | |
| hsa- | MI0000477 | hsa-miR- | UGAGAACUGAAUUCCAUGGGUU | 3 |
| miR- | | 146a-5p | | |
| 146a | | | | |
| hsa- | MI0000270 | hsa-miR- | AACAUUCAUUGCUGUCGGUGGGU | 4 |
| miR- | | 181b-5p | | |
| 181b-1 | | | | |

The inhibitors used herein can comprise antisense oligonucleotides comprising a sequence that is complementary to at least 80%, 85%, 90%, 95%, or 100% of the consecutive length of the target miRNA. For example, the hsa-miR-145-5p inhibitor can comprise at least 16, 17, 18, 19, 20, 21, or all 22 consecutive nucleotides of the sequence AGGGATTCCTGGGTTTTCTGGAC (SEQ ID NO:5). As another example, the hsa-miR-143-3p inhibitor can comprise at least 16, IT 18, 19, 20, 21, or all 22 consecutive nucleotides of the sequence CAGCTACAGTGCTTCATCTCA (SEQ ID NO:6). In some embodiments, the inhibitors are concatemers of the inhibitory sequences. For example, a number of tandem sponges can be used, e.g., 2, 4, 6, 8, 10, or 12 sponges, or any range therebetween. Linkers or spacers can be used between the sponges, e.g., AATT, as shown in bold in the following examples:

An exemplary sequence with six tandem miR-145 sponges with AATT linkers in bold (152 bp, 5′3):

(SEQ ID NO: 46)
AGGGATTCCTTTTAAACTGGACAATTAGGGATTCCTTTTAAACTGGACAA
TTAGGGATTCCTTTTAAACTGGACAATTAGGGATTCCTTTTAAACTGGAC
AATTAGGGATTCCTTTTAAACTGGACAATTAGGGATTCCTTTTAAACTGG
AC.

An exemplary sequence with six tandem miR-143 sponges with AATT linkers in bold (146 bp, 5′-3′):

(SEQ ID NO: 47)
GAGCTACAGTGCTTCATCTGAAATTGAGCTACAGTGCTTCATCTGAAATT
GAGCTACAGTGCTTCATCTGAAATTGAGCTACAGTGCTTCATCTGAAATT
GAGCTACAGTGCTTCATCTGAAATTGAGCTACAGTGCTTCATCTGA

In some embodiments, the inhibitors comprise Tough Decoy (TuD) RNAs, which comprise of an imperfect RNA hairpin structure that harbors two opposing miRNA binding sites (see, e.g., Hooykaas et al., RNA Biol. 2018; 15(11):1410-1419). An exemplary TuD RNA for miR-143 and miR-145 comprises (5-3′):

(SEQ ID NO: 48)
GACGGCGCTAGGATCATC-AAC-AGGGATTCCTATCTTTTAAACTGG
ACCAA-GTATTCTGGTCAAGAATAC-AAC-GAGCTACAGTATCTGC
TTCATCTGA-CAAGATGATCCTAGCGCCGTC-TTTTTTT.

One, two, three, four, or more TuD RNA encoding sequences can be included.

The miR-143 and miR-145 sponge sequences are in bold font; when a sequence that has less than 100% identity is used, preferably any differences from SEQ ID NO:48 are outside the miR-143 and miR-145 sponge sequences.

The mimics used herein can comprise the mature miRNA sequence, or can comprise a pri-miRNA stem loop sequence, e.g., a miR-146a mimic can comprise CCGATGTGTATCCTCAGCTTTGGAACTGAATTCCATGGGTIGTGTCAGTG TCAGACCTCTGAAATTCACTTCTTCAGCTGGGATATCTCTGTCATCGT (SEQ ID NO:7); a miR-81b-2 mimic can comprise CTGATGGCTGCACTCAACATTCATTYCTGTCGGTGGGITTGAGTCCTGAATC AACTCACTGATCAATCAATGCAAACTGCGGACCAAACA (SEQ ID NO:8). In some embodiments, the mimics are double stranded. In some embodiments, the mimics are single stranded. See, e.g., Wang, Methods Mol Biol. 2011; 676:211-23.

In some embodiments, the mimics are expressed from a miR-146a/miR-181b-2 miRNA mimic expression sequence:

(SEQ ID NO: 43)
TTTAGTAGAGACAAATTCTCCATGTTGCCCAGGCTAGTCCTGAACTCCTGG
GCTCAAGAGATCCACCCACATCAGCCTTCCAGACTGCTGGCCTGGTCTCCT
CCAGATGTTTATAACTCATGAGTGCCAGGACTAGACCTGGTACTAGGAAG
CAGCTGCATTGGATTTACCAGGCTTTTCACTCTTGTATTTTACAGGGCTGG
GACAGGCCTGGACTGCAAGGAGGGGTCTTTGCACCATCTCTGAAAAGCCG
ATGTGTATCCTCAGCTTTGAGAACTGAATTCCATGGGTTGTGTCAGTG
TCAGACCTCTGAAATTCAGTTCTTCAGCTGGGATATCTCTGTCATCGT
GGGCTTGAGGACCTGGAGAGAGTAGATCCTGAAGAACTTTTTCAGTCTGC
TGAAGAGCTTGGAAGACTGGAGACAGAAGGCAGAGTCTCAGGCTCTGAA
GGTATAAGGAGTGTGAGTTCCTGTGAGAAACACTCATTTGATTGTGAAAA
GACTTGAATTCTATGCTAAGCAGGGTTCCAAGTAGCTAAATGAATGATCT
CAGCAAGTCTCTCTTGCTGCTGCTGCTATGAGGTTCTCCAAGCACTCCTTC
CTTCTCTGAACCACAGCTTCCTCATCTGCAATAACCTCCCAGCTCCAATGT
CAATGTTATGGACACCTGTGTGGGCCCTCAATCATGCAGATGGCTGGTTAC
TAAGGGAGAAGCCAGACACACAGACTTCAAAGAACTGAGATGGAAAAGA
AGAGCCAGGAGTCAGCCAGGGAGGGCAAAGGCAACCCCACCAACTGAAA
ACACTGATGGCTGCACTCAACATTCATTGCTGTCGGTGGGTTTGAGTC
TGAATCAACTCACTGATCAATGAATGCAAACTGCGGACCAAACAACCC
AAAGGAAGCAAAAGCATGTGCAGAGAGTCGCCTGAGTGCAGCAGCTACT
GCTGTGAGTGGGAGCTGCTACTATTTCCAAAACACTAGCTAAAGCTTTTCT
TGTTCTCCTTGGTTCTAACATGGAGTAAGTTCTCTAAATTCCTAGTACTTT
TCTCCTTTTTCAATAGTCTAGATTTAAGGAAATGATATTTTATAGGAAGAA
TTAAATACATATCTGGATATCACATCACATTTTAAAAATCAGTA

The miR-146a/miR-181b-2 mimic sequences are in bold font: when a sequence that has less than 100% identity is used, preferably any differences from SEQ ID NO:43 are outside the miR-146a/miR-181b-2 mimic sequences.

In some embodiments of the methods and compositions described herein, variants of any of the nucleic acids described herein can be used that are at least 80%, 85%, 90%, 95%, 97%, 98%, or 99% identical to a sequence provided herein, so long as they retain desired functionality of the parental sequence. In some embodiments, the sequences used can differ by at least or up to 1, 2, 3, 4, or 5 nucleotides (or any range with those values as end points).

To determine the percent identity of two nucleic acid sequences, the sequences are aligned for optimal comparison purposes (e.g., gaps can be introduced in one or both of a first and a second amino acid or nucleic acid sequence for optimal alignment and noni-homologous sequences can be disregarded for comparison purposes). In a preferred embodiment, the length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

Provided herein are compositions comprising the transdifferentiation factors, e.g., as RNAs, e.g., individual RNAs or as an RNA concatemer, or sequences encoding the transdifferentiation factors, e.g., as DNA, e.g., in an expression construct. The RNA concatemers can comprise one or more copies of each of the transdifferentiation factors, with optional linkers therebetween.

In some embodiments the RNAs are modified A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides, Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages.

In some embodiments, the RNAs include a cholesterol moiety, e.g., at the 3′-end. In some embodiments, the RNAs have various modifications for RNase protection and pharmacologic properties such as enhanced tissue and cellular uptake. For example, in addition to the modifications discussed above for antisense oligonucleotides, an RNA can have one or more of complete or partial 2′-O-methylation of sugar and/or a phosphorothioate backbone. Phosphorothioate modifications provide protection against RNase activity and their lipophilicity contributes to enhanced tissue uptake. In some embodiments, the RNA cam include six phosphorothioate backbone modifications; two phosphorothioates are located at the 5-end and four at the 3-end. See, e.g., Krutzfeldt (2005) Nature 438, 685-689; Czech (2006) N Engl J Med, 354:1194-1195; Robertson (2010) Silence. 1:10; Marquez and McCaffrey (2008) Hum Gene Ther., 19(1):27-38; van Rooij (2008) Circ Res. 103(9):919-928; and Liu (2008) Int. J. Mol. Sci. 9:978-999; (Ebert (2010) RNA 16, 2043-2050). RNAs useful in the present methods can also be modified with respect to their length or otherwise the number of nucleotides making up the RNA. The RNAs must retain specificity for their target, i.e., must not directly bind to, or directly significantly affect expression levels of, transcripts other than the Intended target.

In some embodiments, the inhibitory RNA is locked and includes a cholesterol moiety (e.g., a locked antagomir; Krutzfeldt (2005) Nature 438, 685-689).

One or more substituted sugar moieties can also be included, e.g., one of the following at the 2′ position: OH, SH, SCH₃, F, OCN, OCH₃, OCH₃ O(CH₂)ₙ CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)](Martin (1995) Helv. Chim. Acta 78, 486). Other preferred modifications include 2′-methoxy (2′-0-CH₃), 2-propoxy (2′-OCH₂ CH₂CH₃) and 2′-fluoro (2′-F). Similar modifications may also be made at other positions on the oligonucleotide, particularly the 3 position of the sugar on the 3 terminal nucleotide and the 5′ position of 5′ terminal nucleotide. Oligonucleotides may also have sugar mimetics such as cyclobutyls in place of the pentofuranosyl group.

The nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentiobiosyl HMC, as well as synthetic nucleobases, e.g., 2-aminoadenine, 2-(methylamino)adenine, 2-(imidazolylalkyl)adenine, 2-(aminoalkylamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, 2,6-diaminopurine; 5-ribosyluracil (Carlile (2014) Nature 515(7525): 143-6). Komberg, A., DNA Replication, W. H. Freeman & Co., San Francisco, 1980, pp 75-77; Gebeyehu (1987) Nucl Acids Res. 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide. In some embodiments, both the nucleobase and backbone may be modified to enhance stability and activity (El-Sagheer (2014) Chem Sci 5:253-259)

In some embodiments, both a sugar and an internucleoside linkage, i.e., the backbone, of the nucleotide units are replaced with novel groups.

In some embodiments, the inhibitor is a miRNA sponge or a variation of miRNA sponge, such as target mimics (Franco-Zorrilla (2007) Nat Genet 39:1033-1037), decoys (Care (2007) Nat Med 13:613-618, miRNA target sequences (Gentner (2009) Nat Methods 6:63-66), miRNA erasers (Saved (2008) Mol Biol Cell 19:3272-3282), and lentivirus-mediated antagomirs (Scherr (2007) Nucleic Acid Res 35:e149). Sponge constructs typically contain 4-10 binding sites separated by a few nucleotides each. The efficacy of miRNA sponges depends on affinity and avidity of binding sites, as well as the concentration of sponge RNAs relative to the concentration of the miRNA.

Also provided are composition comprising the transdifferentiation factors as DNA, e.g., in an expression construct. Exemplary expression constructs can include sequences encoding the transdifferentiation factors in a naked DNA construct, plasmids, or viral vectors, including recombinant retroviruses, adenovirus, adeno-associated virus, lentivirus, and herpes simplex virus-1, or recombinant bacterial or eukaryotic plasmids. In some embodiments, the DNA expression constructs can include a promoter sequence, e.g., a constitutive promoter (e.g., human β-actin, human elongation factor-1α, chicken β-actin combined with cytomegalovirus early enhancer, cytomegalovirus (CMV), simian virus 40, and herpes simplex virus thymidine kinase) or a smooth muscle cell-specific promoter or enhancer sequence (e.g., a promoter from ACTA2 (α-smooth muscle actin), MYH11 (smooth muscle myosin heavy chain; formerly known as SM-MHC), CNN1 (calponin), TAGLN (transgelin; also referred to herein as SM22a), CALD1 (h-caldesmon), and SMTN (smoothelin)), or U6 promoter sequence; an enhancer sequence, e.g., woodchuck hepatitis virus post-transcriptional regulatory element (WPRE); 5′ untranslated region (UTR), a 3′ UTR; a polyadenylation site; and/or an insulator sequence.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging; virus lines for preparing both ecotropic and amphotropic retroviral systems include ψCrip, ψCre, ψ2 and ψAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad, Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al (1991) Proc. Natl. Acad. Sci. USA 88:8377-8381; Chowdhury et al. (1991) Science 254:1802-1805; van Beusechem et al. (1992) Proc. Natl. Acad, Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover. the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc, Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). In some instances, the AAV vector is selected from the group consisting of AAV1, AAV2, AAV3, AAV4, AAV5, AAV6, AAV7, AAV8, AAV9, AAVrh10, AAV11, and AAV12. In sone embodiments, AAV2, AAV2.5 (Lompré et al., Gene Ther. 2013 September; 20(9): 901-912), or AAV9 is used.

A vector as described herein can be a pseudotyped vector. Pseudotyping provides a mechanism for modulating a vector's target cell population. For instance, pseudotyped AAV vectors can be utilized in various methods described herein. Pseudotyped vectors are those that contain the genome of one vector, e.g., the genome of one AAV serotype, in the capsid of a second vector, e.g., a second AAV serotype. Methods of pseudotyping are well known in the art. For instance, a vector may be pseudotyped with envelope glycoproteins derived from Rhabdovirus vesicular stomatitis virus (VSV) serotypes (e.g., Indiana and Chandipura strains), rabies virus (e.g., various Evelyn-Rokitnicki-Abelseth ERA strains and challenge virus standard (CVS)). Lyssavirus Mokola virus, a rabies-related virus, vesicular stomatitis virus (VSV), Mokola virus (MV), lymphocytic choriomeningitis virus (LCMV), rabies virus glycoprotein (RV-G), glycoprotein B type (FuG-B), a variant of FuG-B (FuG-B2) or Moloney murine leukemia virus (MuLV). A virus may be pseudotyped for transduction of one or more neurons or groups of cells.

Without limitation, illustrative examples of pseudotyped vectors include recombinant AAV2/1, AAV2/2, AAV2/5, AAV2/6, AAV2/7, AAV2/8, AAV9, AAVrh10, AAV11, and AAV12 serotype vectors. In particular instances, the present disclosures can include a pseudotyped AAV viral vector including a nucleic acid as disclosed herein. See Viral Vectors for Gene Therapy: Methods and Protocols, ed. Machida, Humana Press, 2003. Modified vectors can also be used, e.g., vectors in which peptides EYHHYNK (EYH) and GETRAPL (GET) are incorporated into the HI loop of the capsid protein of adeno-associated virus-2 (AAV-2) (Work et al., Mol Ther. 2004 February; 9(2):198-208).

In Vitro Conversion of SMCs or Fibroblasts to iECs

The present methods can include the use of SMCs or fibroblasts that are treated in vitro to induce transdifferentiation to iECs. In some embodiments, the SMCs or fibroblasts are isolated from a subject, e.g., a human or non-human veterinary subject (preferably the subject is a mammal). For example, human vascular smooth muscle cells can be isolated from multiple tissue types including aorta (AoSMC) coronary artery (CASMC), pulmonary artery (PASMC), umbilical artery (UASMC), bladder smooth muscle cells, (HBdSMC), adipose tissue smooth muscle cells (ASMCs), or blood derived circulating smooth muscle cell progenitors (SPCs). For fibroblasts, human fibroblasts can be isolated from multiple tissue types including skin (dermal fibroblasts), lung fibroblasts, cardiac fibroblasts, aortic fibroblasts, adipose tissue fibroblasts, or foreskin fibroblasts. Methods for isolating SMCs or fibroblasts are known in the art; see, e.g., Lu et al., J Cardiothorac Surg. 2013 Apr. 12; 8:83 (isolation from human aortic dissection); Ribeiro et al., J Vis Exp. 2010; (41): 1940 (isolation of Human Umbilical Arterial Smooth Muscle Cells (HUASMC)); Patel et al., (2016). Isolation, Culture, and Characterization of Vascular Smooth Muscle Cells. In: Martin, S., Hewett, P. (eds) Angiogenesis Protocols. Methods in Molecular Biology, vol 1430. Humana Press, New York, NY. doi.org/10.1007/978-1-4939-3628-1_6: Pustlauk et al., Scientific Reports volume 10, Article number: 5951 (2020); Salemi et al. Stein Cell Research & Therapy (2022) 13:156, doi.org/10.1186/s13287-022-02835-x (Adult stem cell sources for skeletal and smooth muscle tissue engineering); Simper et al., Circulation. 2002; 106:1199-1204 (SMC from progenitors in blood); Ahmetaj-Shala et al., Front Cell Dev Biol. 2021; 9: 681347 (smooth muscle cells grown from circulating blood progenitors); Miao and Li, Br J Pharmacol. 2012 February; 165(3): 643-658 (adipose tissue in vascular smooth muscle cell growth); or Rodriguez et al., Proc Natl Acad Sci USA. 2006 Aug. 8; 103(32):12167-72 (multipotent stem cells in human adipose tissue differentiate into functional smooth muscle cells). Alternatively, the SMCs or fibroblasts can be obtained commercially, e.g., from Lonza, Cell Biologics, Lifeline Cell Tech, Thermo Fisher. or ATCC. For therapeutic applications, the SMCs or fibroblasts are preferably obtained from the subject to be treated, i.e., the SMCs or fibroblasts are autologous.

In preferred embodiments, the SMCs express a smooth muscle actin (SMA), vimentin, calponin and/or myosin heavy chain 11 (MYH11). In some embodiments, the methods include detecting the expression of, and optionally purifying a population of SMCs, based on expression of αSMA, vimentin, calponin, and/or MYH11, e.g. using FACS.

In some embodiments, the SMCs are transfected with the constructs described herein, e.g., as RNA, e.g., individual RNAs or as an RNA concatemer. or as DNA, e.g., in an expression construct as described above. Expression constructs can be administered in any effective carrier, e.g., any formulation or composition capable of effectively delivering the component gene to cells in vivo. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vivo. The transdifferentiation factors can be in contact with the cells for a time sufficient for a desired percentage of the cells to be transfected, e.g., 2-72 hours, e.g., 12-48 hours, e.g., at least 2, 4, 6, 8, 10, 12, 24, 36, or 48 hours, up to 48 or 60 hours or more.

During this time, reprogramming occurs and thus after transfection, the cells are grown in supportive media, optionally supplemented with a protein Kinase A (PKA) activator, e.g., 8-Br-cAMP, aid a TGFβ-RI antagonist, e.g., SB 431542 (e.g., about 2 μL/mL, 10 μM stock solution, Stem Cell). The media can be, e.g., endothelial cell growth media such as EGM-2 media from Lonza comprising basal media and growth factors, e.g., hydrocortisone, hFGF-B, VEGF, R3-IGF-1, ascorbic acid, hEGF, GA-1000, and heparin, or EBM media from Lonza. In some embodiments, the supportive media comprises Bovine Brain Extract-Hammond (BBE). Other endothelial cell growth media formulations are known in the art and/or are commercially available, e.g., from Sigma-Aldrich, R&D systems, or promocell. The PKA activator can be, e.g., 8-Bromo-cAMP; Dibutyryl-cAMP; 8-CPT-cAMP; Taxol; Adenosine 3′,5-cyclic Monophosphate, N6-Benzoyl-, Sodium Salt: Adenosine 3′,5′-cyclic monophosphate; Belinostat; Adenosine 3′,5′-cyclic monophosphate sodium salt monohydrate; (S)-Adenosine, cyclic 3prime,5prime-(hydrogenphosphorothioate) triethylammonium; Sp-Adenosine 3prime,5prime-cyclic monophosphorothioate triethylammonium salt; Sp-5,6-DCI-cBiMPS; 8-Bromoadenosine 3′,5′-cyclic Monophosphothioate, Sp-Isomer sodium salt; Adenosine 3prime,5prime-cyclic Monophosphorothioate, 8-Bromo-, Sp-Isomer, Sodium Salt; Sp-8-pCPT-cyclic GMPS Sodium; 8-Bromoadenosine 3′,5′-cyclic monophosphate; N6-Monobutyryladenosine 3prime:5prime-cyclic monophosphate sodium salt; 8-PIP-cAMP; or Sp-cAMPS, all of which are known in the art or commercially available, e.g., from Tocris or R&D Systems. The TGFβRI antagonist can be, e.g., SB 431542; A83-01; RepSox; SB 525334; SB 505124; Galunisertib; LY 364947; LY2109761; LY3200882; LY573636; LY364937; Ki26894; LY580276; SB-431542; SB-505124; SD-093; SD-208; IN-4130; Vactosertib; D 4476; GW 788388; SD 208; R 268712; IN 1130; SM 16; A 77-01; or AZ 12799734, all of which are known in the art or commercially available, e.g., from Tocris or R&D Systems.

The cells can be grown, e.g., for at least 2, 4, 6, 8, 10, 12, 14, 16, 18, or 20 days, until a desired number of cells has been reached.

The methods can then include sorting for cells that express ICAM-1, kinase insert domain receptor (KDR), Vascular endothelial growth factor receptor 2 (VEGFR2), and/or von Willebrand factor (vWF), followed by expansion of the sorted cells, e.g., ICAM-1+ cells.

In Vivo Conversion of SMCs to iECs

In some embodiments, iECs can be generated from cardiac SMCs in vivo using direct delivery approaches, e.g., delivery in a viral vector as described herein. In these methods the transdifferentiation factors, or sequences encoding the transdifferentiation factors, are administered to a subject.

A preferred approach for in vivo introduction of nucleic acid into a cell is by use of a viral vector containing an expression construct as described herein, or using RNA. Infection of cells with a viral vector has the advantage that a large proportion of the targeted cells can receive the nucleic acid. Additionally, molecules encoded within the viral vector. e.g., by a cDNA contained in the viral vector, are expressed efficiently in cells that have taken up viral vector nucleic acid.

In addition to viral transfer methods, such as those illustrated above, non-viral methods can also be employed to cause expression of a nucleic acid compound described herein (e.g. a nucleic acid encoding a transdifferentiation factors) in the tissue of a subject. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest, Dermatol. 116(1):131-135 (2001); Cohen et al., Gene Ther. 7(22):1896-905 (2000); or Tarn et al., Gene Ther. 7(21):1867-74 (2000).

In some embodiments, a sequence(s) encoding the transdifferentiation factors is entrapped in liposomes bearing positive charges on their surface (e.g., lipofectins), which can be tagged with antibodies against cell surface antigens of the target tissue (Mizuno et al., No Shinkei Geka 20:547-551 (1992): PCT publication WO91/06309; Japanese patent application 1047381; and European patent publication EP-A-43075).

In clinical settings, the gene delivery systems for the transdifferentiation factors can be introduced into a subject by any of a number of methods, each of which is familiar in the art. For instance, a pharmaceutical preparation of the transdifferentiation factor delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., PNAS USA 91: 3054-3057 (1994)).

A pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### Methods of Treatment

Further provided herein are methods for the treatment of endothelial injury, e.g., treatment of subjects undergoing a cardiovascular intervention, e.g., to treat and reduce risk of cardiovascular health issues like heart attacks and coronary artery disease. Cardiovascular interventions can include angioplasty, stent placement, catheter ablation, heart valve surgery, or bypass surgery. The methods can also be used for the treatment of disorders associated with ischemic injury. In some embodiments, the disorder is myocardial infarction, ischemic stroke, ischemic renal injury, limb ischemia, arteriovenous (AV) fistula injury, organ transplant graft injury, or wound healing. As used in this context, to “treat” means to ameliorate at least one symptom of the disorder.

Generally, the methods include administering a therapeutically effective amount of a treatment as described herein, to a subject who is in need of, or who has been determined to be in need of, such treatment. The treatment can include administering iECs obtained by in vitro conversion methods described herein, e.g., by intravenous or intra-arterial infusion, or by administering the transdifferentiation factors for in vivo conversion as described herein.

### Compositions and Methods of Administration

Provided herein are compositions, including pharmaceutical compositions comprising or consisting of iECs or transdifferentiation factor expression constructs as an active ingredient, as well as methods of use thereof.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols. glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid: buffers such as acetates, citrates or phosphates and agents for the adjustment of tonicity such as sodium chloride or dextrose, pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g. as described in U.S. Pat. No. 6,471,996).

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Materials and Methods

The following materials and methods were used in the examples herein.

### Cell Culture

Human Umbilical Vein Endothelial Cells (HUVECs, Lonza), Human Aortic Endothelial Cells (HAECs, Lonza), and iECs were cultured in endothelial cell growth media (EGM-2, Lonza). Human Coronary Artery Endothelial Cells (CAECs, Lonza), were cultured in microvascular EGM-2 growth media (ECM-2 MV, Lonza). Human Coronary Artery Smooth Muscle Cells (CASMCs, Lonza) were cultured in smooth muscle cell growth media (SMGM, Lonza) prior to use.

### Transdifferentiation Protocol

CASMCs were seeded into 6-well plates 24 hours prior to transfection at 60% confluency. Transfection was performed using Lipofectamine RNAiMAX (Invitrogen) according to manufacturer specifications. Briefly, miR-143-3p (Ambion/ThermoFisher) and miR-145-5p (Ambion/ThermoFisher) inhibitors and miR-146a-5p (Ambion/ThermoFisher) and miR-181b-5p (Ambion/ThermoFisher) mimics were used for transfection at 50 nM. MiRNA inhibitors and mimics were mixed with OptiMEM and mixed with RNAiMAX in OptiMEM (Gibco) for 5 minutes to allow for lipid complexing. The transfection solution was then added to each well and incubated for 48 hours. After 48 hours, SMGM was aspirated and EGM-2 with 8-Br-cAMP (2 μL/2 mL, 0.1 mM stock solution, Sigma-Aldrich) and SB 431542 (2 μL/mL, 10 μM stock solution, Stein Cell) was used to facilitate expansion of the induced endothelial cells. Cells were expanded for 12 days prior to fluorescence-activated cell sorting for intercellular adhesion molecule-1 (ICAM-1 HA58 Biolegend, APC- or PE-conjugated). Sorted cells were expanded in plates using the above EGM-2 conditions for analysis and experimental use.

### RT-qPCR

Total RNA was extracted with QIAzol or TRIzol reagent and miRNeasy micro kit (Qiagen) per manufacturer protocol. RNA concentration and quality were assessed using Qubit (ThermoFisher) and Bioanalyzer (Agilent). Isolated RNA was reverse transcribed using a High-Capacity cDNA Reverse Transcription kit (Applied Biosystems) or miRCURY LNA miRNA RT kit (Qiagen) accord to manufacturer specifications. Real-time quantitative PCR (RT-qPCR) was conducted on the AriaMx RT-qPCR system (Agilent). Specific primers include ACTA2, calponin, MYH11, vimentin, VE-Cadherin, CD31, KDR, vWF, SELE, VCAM1, and GAPDH were purchased (Life Technologies). All expression values were normalized to GAPDH and represent 2^{-ΔCt}. The primers used were as shown in the table below. For miRNAs, miRCURY LNA miRNA PCR primers (Qiagen) for miR-143-3p, miR-145-5p, miR-146a-5p, and miR-181b-5p were used.

TABLE A
Primers

| Gene | Primer 5′-3′ | # | Primer 5′-3′ | # |
|---|---|---|---|---|
| ACTA2 | AAAAGACAGCTACGTGGGTGA | 9. | GCCATGTTCTATCGGGTACTTC | 10. |
| calponin | CTGTCAGCCGAGGTTAAGAAC | 11. | GAGGCCGTCCATGAAGTTGTT | 12. |
| MYH11 | CGCCAAGAGACTCGTCTGG | 13. | TCTTTCCCAACCGTGACCTTC | 14. |
| vimentin | GACGCCATCAACACCGAGTT | 15. | CTTTGTCGTTGGTTAGCTGGT | 16. |
| VE-Cadherin | TTGGAACCAGATGCACATTGAT | 17. | TCTTGCGACTCACGCTTGAC | 18. |
| CD31 | AACAGTGTTGACATGAAGAGCC | 19. | TGTAAAACAGCACGTCATOCTT | 20. |
| KDR | GGCCCAATAATCAGAGTGGCA | 21. | CCAGTGTCATTTCCGATCACTTT | 22. |
| vWF | CCGATGCAGCCTTTTCGGA | 23. | TCCCCAAGATACACGGAGAGG | 24. |
| SELE | AGAGTGGAGCCTGGTCTTACA | 25. | CCTTTGCTGACAATAAGCACTGG | 26. |
| VCAM1 | GGGAAGATGGTCGTGATCCTT | 27. | TCTGGGGTGGTCTCGATTTTA | 28. |
| BMPR2 | CGGCTGCTTCGCAGAATCA | 29. | TCTTGGGGATCTCCAATGTGAG | 30. |
| TGFBR1 | ACGGCGTTACAGTGTTTCTG | 31. | GCACATACAAACGGCCTATCTC | 32. |
| TGFBR2 | GTAGCTCTGATGAGTGCAATGAC | 33. | CAGATATGGCAACTOCCAGTG | 34. |
| Activin | AGACACGGGAGTGCATCTACT | 35. | GCCTATCGTAGCAGTTGAAGTC | 36. |
| GAPDH | GGAGCGAGATCCCTCCAAAAT | 37. | GGCTGTTGTCATACTTCTCATGG. | 38. |

#, SEQ ID NO:

### Western Blot

Cultured cells were harvested and lysed in RIPA buffer (50 mM Tris-HCL pH 7.4, 150 mM NaCl, 1% NP-40, 0.5% sodium deoxycholate, 0.1% SDS) in combination with protease inhibitor cocktail tablets (Roche) and phosphatase inhibitors (Invitrogen). Protein concentrations were determined using a Pierce BCA Assay (ThermoFisher) and protein was loaded on gradient 4-20% Mini-PROTEAN TGX precast gels (Bio-Rad). Separated proteins were transferred to PVDF membranes using the Transfer Turbo Blot System (Bio-Rad) and Trans-Blot Turbo RTA Transfer kit (Bio-Rad) for 2.5 minutes. Membranes were then blocked with 2.5% BSA in TBST for 1 hour at room temperature before being incubated at 4° C. overnight with primary antibodies against ICAM-1 (HA-58, YN1/1.7.4, Biolegend), VCAM1 (H-276, Santa Cruz Biotechnology, 1:1000), SELE (H-300, Santa Cruz Biotechnology, 1:1000), vWF (1.B 690, Santa Cruz Biotechnology, 1:1000), CD3l (D8VJE, 89C2, Cell Signaling, 1:1000; EPR17569, Abcam, 1:250), VE-Cadherin (D87F2, Cell Signaling, 1:1000; VECD1, Biolgend, 1:1000), eNOS (M221, ABCAM, 1:1000), pSer-1177 eNOS (EPR20991, ABCAM, 1:1000), MYH11 (MYH11/23, 1:1000, ABCAM), vimentin (EPR3776, ABCAM, 1:1000), αSMA (D4K9N, Cell Signaling, 1:1000), calponin (EP798Y, ABCAM, 1:1000), and GAPDH (14C10, Cell Signaling, 1:1000). For eNOS and pSer-177 eNOS, samples were incubated with 50 ng/nil VEGF (R&D Systems) for indicated time points. Membranes were washed in TBST and incubated with HRP-conjugated anti-mouse (Cell Signaling, 1:5000), anti-goat (Cell Signaling, 1:5000), and anti-rabbit (Cell Signaling, 1:5000) secondary antibodies for 1 hour at room temperature. Visualization of protein bands was performed using SuperSignal West Femto Maximum Sensitivity Substrate (Life Technologies) solution and a luminescent image analyzer (Bio-Rad, Chemidoc). Densiometric analysis was analyzed in ImageJ (NIH). All measurements were normalized to GAPDH (Cell Signaling, 1:1000) lanes run for each sample.

### Immunofluorescent Staining and Imaging

For confocal analysis, each sample was fixed in 4% PFA (Boston Bio Products) for 30 minutes and then permeabilized with 0.5% Triton-X and blocked with 2.5% BSA for 1 hour. Coverslips were then incubated with primary antibodies against KDR (7D4-6, Biolegend, 1:100), VE-Cadherin (BV9, Biolegend, 1:200), CD31 (WM59, Biolegend, 1:100), vimentin (EPR3776, ABCAM, 1:100), MYH11 (MYH11/23, ABCAM, 1:100), or αSMA (EPR5368, ABCAM, 1:100) overnight. After washing, samples were incubated with the appropriate Alexafluor 488-conjugate antibody (1:500) where applicable as well as counter stained with DAPI (Life Technologies, 1:2500). For samples incubated with 1,1′-dioctadecyl-3,3,3′,3′-tetramethylindo-carbocyanine-labelled Acetylated LDL (DiI Ac-LDL, Kalen Biomed) and oxidized LDL (DiI oxLDL, Kalen Biomed). Samples were fixed in 4% PFA (Boston Bio Products) and counter stained with DAPI (Life Technologies, 1:2500). Confocal images were acquired on a Zeiss LSM 880 Confocal inverted microscope at the Beth Israel Deaconess Medical Center confocal imaging and immunohistochemistry core facility) at 40× magnification.

### RNA-Seq Analysis

RNA-Seq analysis was performed after ribodepletion and standard library construction using Illumina HiSeq2500 V4 2x150 PE (Genewiz). All samples were processed using an RNA-seq pipeline implemented in the bcbio-nextgen project. Raw reads were examined for quality issues using FastQC (bioinformatics.babraham.ac.uk/projects/fastqc/) to ensure library generation and sequencing were suitable for further analysis. Trimmed reads were aligned to UCSC build mm10 of the mouse genome and augmented with transcript information from Ensenmbl releases 86 (H. sapiens) using STAR. Alignments were checked for evenness of coverage, rRNA content, genomic alignment context and other quality checks using a combination of FastQC and Qualimap. Counts of reads aligning to known genes were generated by featureCounts. Differential expression at the gene level was called with DESeq2. Total gene hit counts and CPM values were calculated for each gene and downstream differential expression analysis between specified groups was performed using DESeq2 and an adapted DESeq2 algorithm that excludes overlapping reads. Genes with adjusted p-value<0.05 and Log₂(FC)>2.0 were labeled as differentially expressed genes for each comparison. The mean quality score of all samples was 35.81 with a range of 37,000,000-53,000,000 reads per sample. All samples had >93% of mapped fragments over total fragments. Differentially expressed genes (DEGs) were identified as being at least 2.0-fold change and adjusted p-value<0.05. DEGs were subjected to gene set enrichment analyses by using MetaCore™ (Clarivate) software. Enrichment analysis for functional ontologies (Process Networks) and analysis using network building tools was performed in MetaCore™. A false discovery rate (FDR)<0.05 was used as threshold for significance in enrichment analysis. Visualization of pathway enrichment analysis were performed as dotplot using (ggplot2 package) and circus plot (circlize package) in K program (18). The function pheatmap in the K package ggplots was used to generate the hierarchical clustering and the associated heat maps for RNA-Seq data. Pairwise correlation matrix between items was created based on Euclidean distance matrix and graphical display using corrplot R package, with the smallest distances in red and the largest distances in blue.

For identification of predicted targets of the 4-miRNA combination, differentially expressed genes (adjusted P<0.05) were used to obtain miRNA-target interactions according to IPA microRNA Target Filter tool (IPA winter release December 2020). Different miRNA target prediction programs (TargetScan, miRecords, ingenuity Knowledge Base and TarBase) filtered our miRNA-target pairings. Confidence filter was used by selecting both experimentally observed and predicted target correlations. Up-regulated miRNA targets of miR-143-3p and/or miR-145-5p, and down-regulated miRNA targets of miR-146a-5p and miR-181b-5p were then used to perform pathway analysis using Metacore.

Microarray Analysis and miRNA Screening

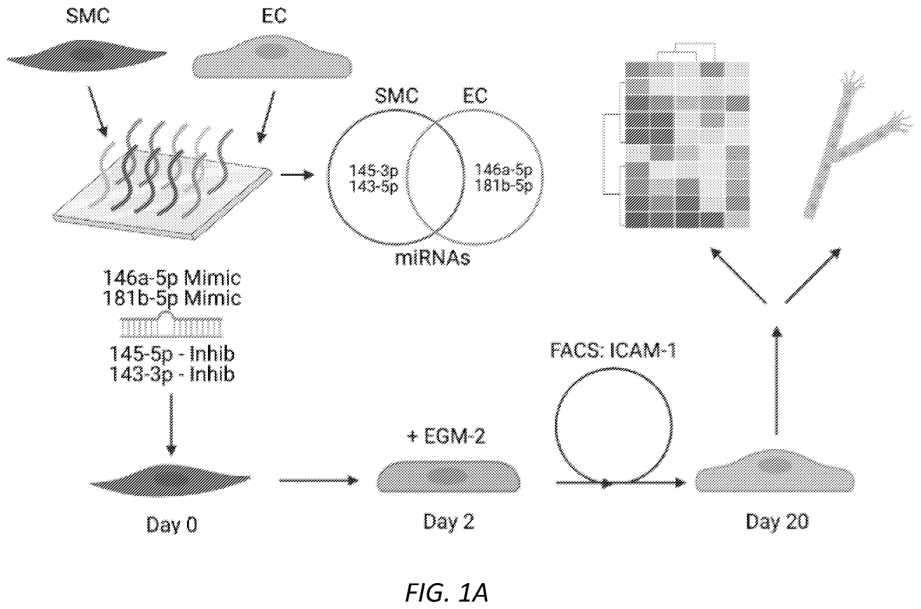

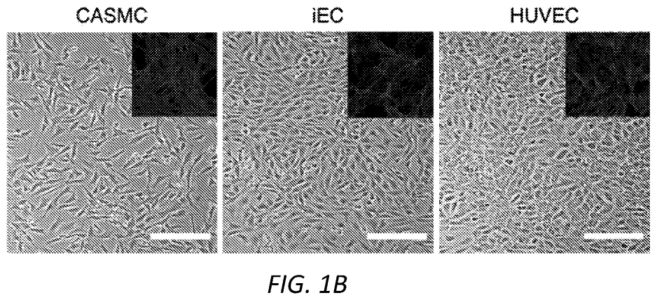

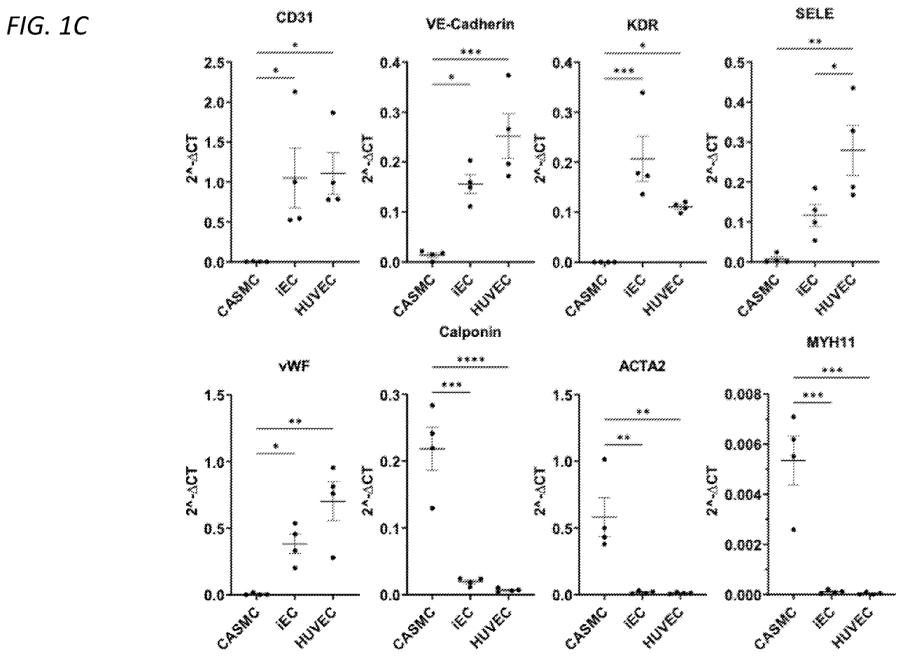

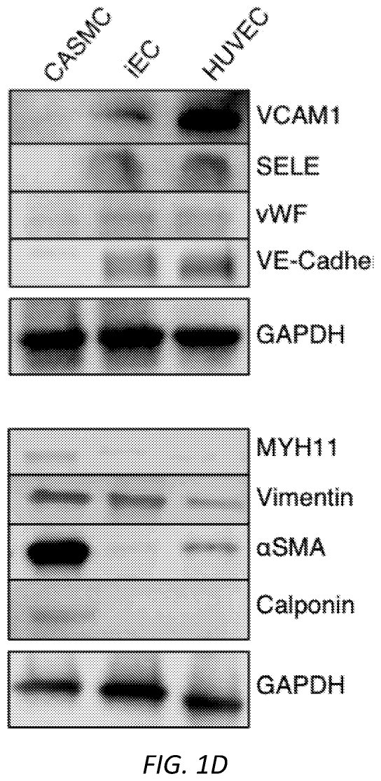

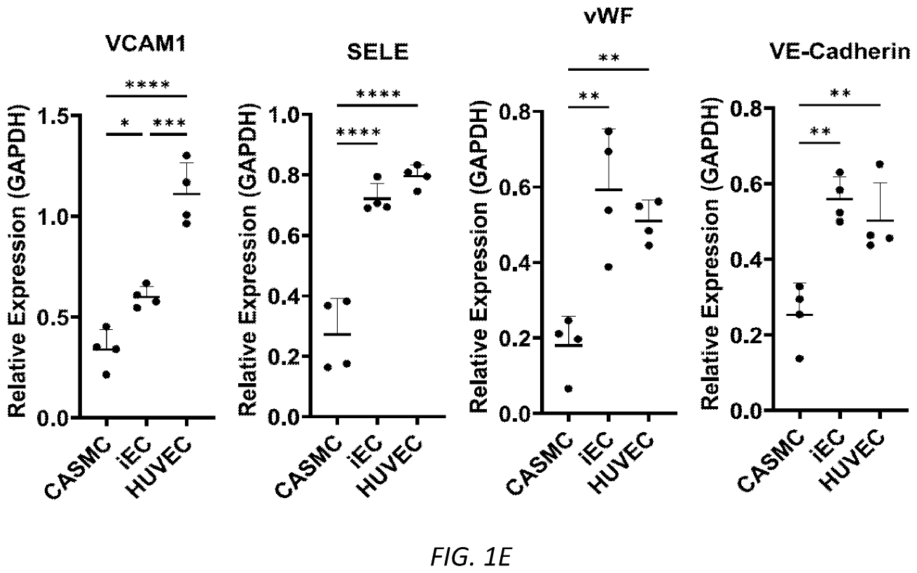

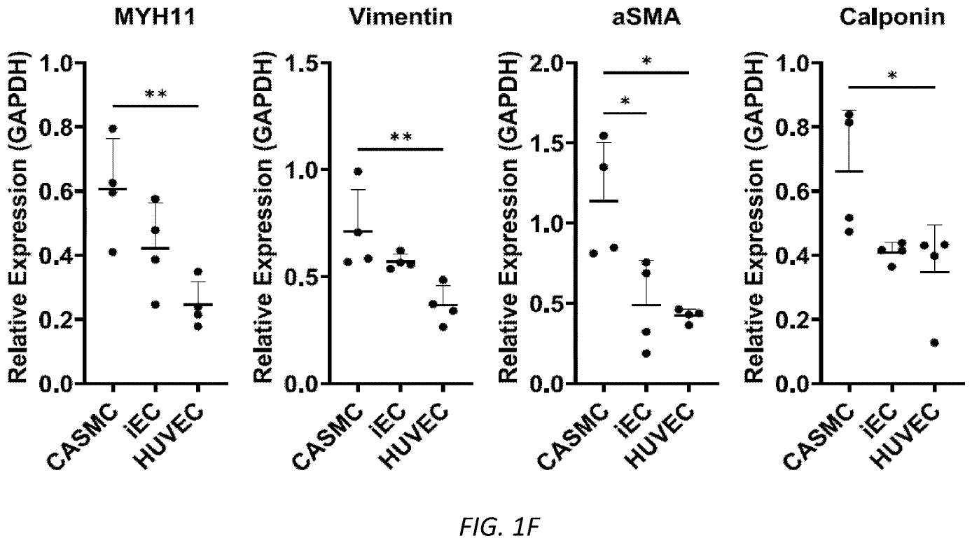

Human coronary artery endothelial cells (CAECs) and human aortic smooth muscle cells (HAoSMCs) were sent to LC Sciences© for miRNA expression profiling analysis. Significant genes were identified as those whose values met the cutoff range at a P_{adj}<0.05 and Log₂(FC)>1. Top differentially regulated miRNAs were identified and miR-143-3p (Ambion), miR-145-5p (Ambion), and miR-214-5p (Ambion) inhibitors and miR-126-5p (Ambion), miR-146a-5p (Ambion), and miR-181b-5p (Ambion) mimics were assessed. Expression of selected markers was assessed after 48 hours of transfection at 50 nM in Lipofectamine 2000 via Western blot. Samples were normalized to beta-actin (Cell Signaling, 1:10000). FIG. 1A schematic was generated with BioRender (BioRender.com).

### In Vitro Functional Assays

Matrigel™ basement membrane matrix was added to 24-well culture plates and incubate at 37° C. for 30 minutes to allow for gelation, HUVEC, CASMC, and iECs were plated at 2×10⁴ cells/well and network formation was assessed every 4 hours for 16 hours. Images were captured with a CytoSMART Omni. Network formation was quantitated by counting the number of tubes formed per field of view. Transwell migration assays were performed by seeding 2.5×10⁴ cells/insert (8 μm pore, Corning) in 200 μL of media. The chamber was flushed with 500 μL of basal media overnight prior to treatment with 50 ng/mL of VEGF. Samples were fixed in 4% PFA (Boston Bio Products) and the underside stained with DAPI (Life Technologies, 1:2500). For BrdU assays, 5×10³ cells were seeded per 96-well plate and allowed to adhere overnight. Samples were then incubated with BrdU-incorporating reagent for 12 hours, with and without 50 ng/mL of VEGF (R&D Systems). Samples were then fixed and quantitated using the Cell Proliferation ELISA BrdU Colorimetric kit accord to manufacturer instructions (Sigma-Aldrich)

### Animal Studies

All protocols concerning animal use were approved by the Institutional Animal Care and Use Committee at Brigham and Women's Hospital and Harvard Medical School, Boston, MA. USA and conducted in accordance with the National Institutes of Health (NIH) Gide for the Care and Use of Laboratory Animals.

### Hindlimb Ischemia and Cell Injection

Ischemic injury of the hindlimb was induced through a unilateral femoral artery ligation in NOD SCID mice. Mice were anesthetized and the femoral artery with surrounding tissue was sutured closed at the proximal and distal ends and cauterized. Immediately following surgery, mice were imaged on a Laser Doppler Imager-2, and were re-imaged at days 4, 7, 11, and 14. 24 hours after ligation, 100 μL of PBS or 0.5×10⁶ cells/100 μL were injected into the tail vein of each mouse. Visual assessment of limb ischemia was assessed at each imaging session. All blood flow recovery values were generated by comparing the injured limb to the contralateral limb at the same time point.

### Isolation of Primary Mouse Smooth Muscle Cells

Mouse aortas were isolated as described previously (19). Extraction of primary mouse cells was accomplished via tissue digestion and magnetic bead isolation. Briefly, isolated aortas were then minced into smaller sections and then placed into 1 mL of 1 mg/ml collagenase IV and dispase for 45 minutes and then resuspended in 10 mL of 0.4% BSA-DPBS to halt digestion. Samples were then filtered with a 70 μm filter and resuspended in 10 mL of 0.4% BSA-DPBS prior to centrifugation. After centrifugation, cells were resuspended in BSA-DPBS. This cell suspension was then stained with anti-CD90.2-PE (fibroblasts, 1:100) or anti-CD31-PE (endothelial cells, 1:100) for 20 minutes at 4° C. Cells were spun down and centrifuged with anti-PE MicroBeads (Miltenyl Biotech, 1:5) for 20 minutes at 4° C. Labeled cell suspensions were spun down, washed, and passed through a magnetic column, capturing CD-90.2-labeled cells. The magnetic source was then removed, the effluent collected, and cells plated and expanded for future use. ECs from muscle and heart were extracted utilizing similar approaches.

### Statistics

Statistical analyses were performed using GraphPad Prism 9.1 (GraphPad Software Inc.). A Student's t-test, one-way, or two-way ANOVA with Tukey's post-hoc test was used to determine significance where appropriate. Data and error bars represent mean±SEM. A P value≤0.05 was considered statistically significant.

### Example 1. Direct Reprogramming of Vascular Smooth Muscle Cells into iECs with miRNAs

miRNAs have emerged as key regulators of cell phenotype and lineage commitment. We sought to identify a combination of miRNAs whose expression profile was indicative of smooth muscle or endothelial cells. RNA was isolated from human aortic smooth muscle cells (HAoSMCs) and coronary artery endothelial cells (CAECs), and miRNA differential expression was evaluated via microarray analysis and tabulated, finding 127 differentially expressed miRNAs (FIG. 8A). We reasoned that inhibiting SMC-enriched miRNAs and overexpressing EC-enriched miRNAs may facilitate cellular reprograming of SMCs into inducible endothelial cells (iECs) (FIG. 8B). Of these potential targets, our data identified several miRNAs enriched in SMCs including miR-143-3p, miR-145-5p, and miR-214, and miRNAs enriched in ECs including miR-126-3p, miR-146a-5p, and miR-181-5p that are known to play functional roles in their respective cell types (20-22). MiRNA expression similarity was also confirmed in a separate SMC cell type, coronary artery SMCs (CASMCs) (FIG. 8C). Iterative probing of these targets by Western blot demonstrated that transfection of CASMCs with inhibitors for SMC-enriched miRNAs, miR-143-3p and miR-145-5p, suppressed the expression of SMC markers such as a smooth muscle actin (αSMA) and calponin, whereas transfection of CASMCs with mimics for EC-enriched miRNAs miR-146a-5p and miR-181b-5p increased the expression of EC markers such as kinase insert domain receptor (KDR. VEGFR2) and von Willebrand factor (vWF) compared to the non-specific transfected controls (FIG. 8D-E). Simultaneous transfection of this 4-miRNA combination resulted in the combinatorial outcome of suppression of SMC markers and increased expression of EC markers and this combination was chosen as the final combination moving forward (FIG. 8D-E). We next optimized a more efficient protocol that facilitated expansion of these transdifferentiated cells. After transfection of CASMCs for 2 days at 50 nM for each miRNA, cells were then expanded over 12 days in modified EC growth media (EGM-2) with transforming growth factor β (TGFβ) inhibitor SB-431542 and 8-Bromo-cAMP, which have been previously used to facilitate expansion of endothelial cell types (6, 23-25). Expanded cells were sorted by flow cytometry for intercellular adhesion molecule-1 (ICAM-1)-positivity for each passage, Grown to confluency, this population of iECs, adopt the notable cobblestone pattern of ECs grown in cell culture and is further highlighted from confocal images of f-actin counterstains (FIG. 1B), and were used for subsequent studies. The 1ECs stably expressed EC markers for at least 5 passages without increased expression of SMC markers utilizing this method, Our generated iECs had population-level expression for EC markers CD31 and VEGFR2 as confirmed by flow cytometry (FIG. 1A). To assess possible disease relevance of these miRNAs in ECs, we isolated ECs via magnetic bead fractionation from the heart, aorta, and gastrocnemius of non-diabetic and diabetic mice, a disease known to affect vascular function. RT-qPCR profiling of ECs revealed an overall decrease in the endothelial-enriched miRNAs, miR-146a-5p and miR-181b-5p, in diabetic endothelial cells (FIG. 9A), This same trend was observed in type-2 diabetic human aortic endothelial cells (T2DM HAECs) when compared to HAECs derived from healthy human subjects (FIG. 9B).

### Example 2. Characterization of Endothelial Markers in iECs

We initially evaluated the relative expression of endothelial and SMC markers in iECs by reverse transcription quantitative polymerase chain reaction (RT-qPCR) to assess the relative degree of transformation; CASMCs and human umbilical vein endothelial cells (HUVECs) were used as controls for comparison. These data showed that transfecting CASMCs with the 4-miRNA combination significantly suppressed the transcription of SMC markers actin alpha 2 (ACTA2), calponin, and myosin heavy chain-11 (MYH11) while enhancing the mRNA abundance of VE-Cadherin, CD31, KDR, vWF, and E-selectin (SELE) (FIG. 1C). In line with these results, densitometric analysis of Western blot protein lysates found a significantly decreased abundance of MYH11, vimentin, αSMA, and calponin and an increased abundance of vascular cell adhesion molecule-1 (VCAM-1), SELE, vWF, and VE-Cadherin at the protein level (FIG. 1D-F). We also verified that there was similar EC marker expression between cardiac aortic endothelial cells (CAECs) and iECs as initial microarray profiling utilized CAECs (FIG. 10). Moreover, treatment of CASMCs with SB-431542 and 8-Bromo-cAMP in the absence of the 4-miRNA combination did not significantly induce endothelial cell markers and exhibited nominal effects on SMC markers in control samples. To examine the broader applicability of this approach using other relevant cell types found in the vessel wall, we utilized mouse aortic fibroblasts. After transfection of the 4-miRNA combination in mouse fibroblasts under the same conditions, we confirmed the expression of the endothelial markers VE-cadherin and CD31 in these mouse iECs by Western blot, RT-qPCR, and immunofluorescence (FIG. 11A, L, C.

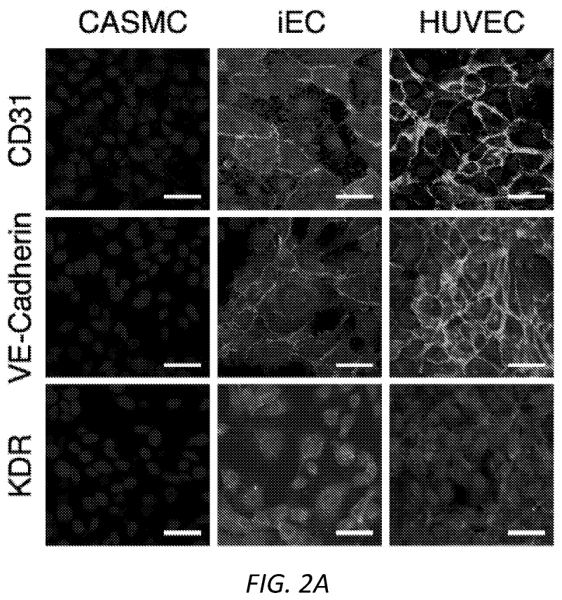

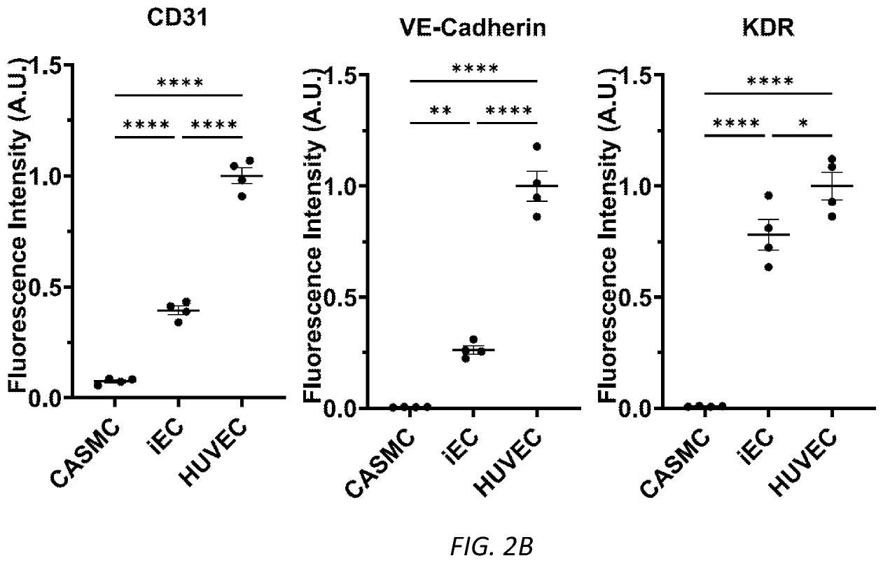

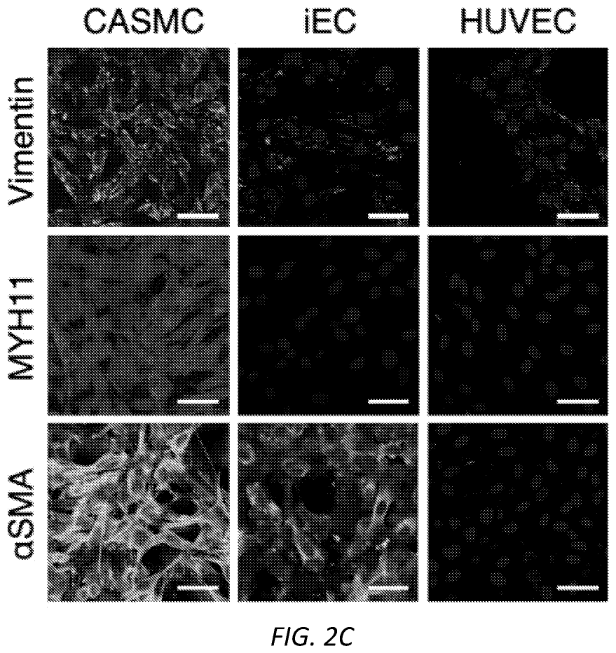

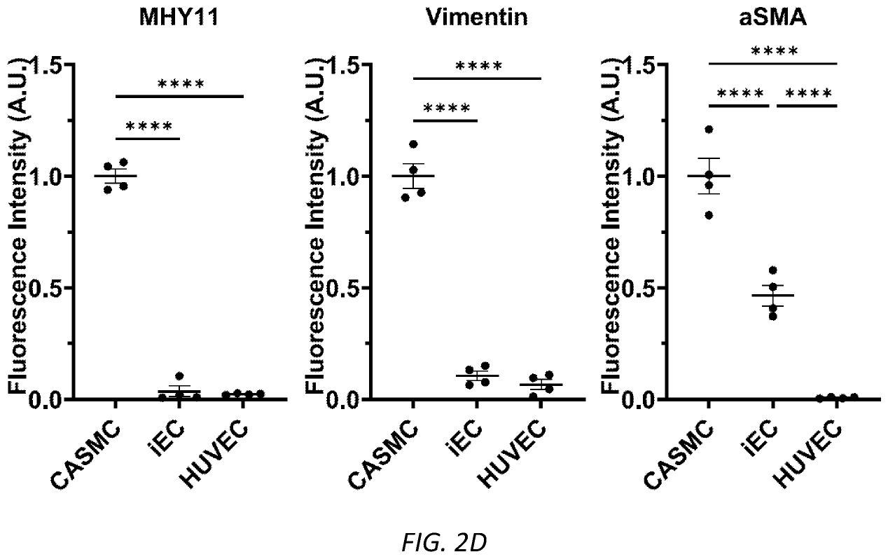

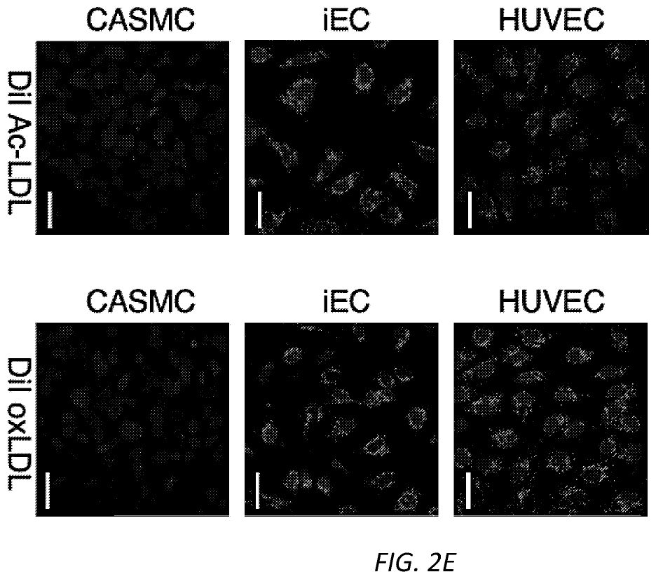

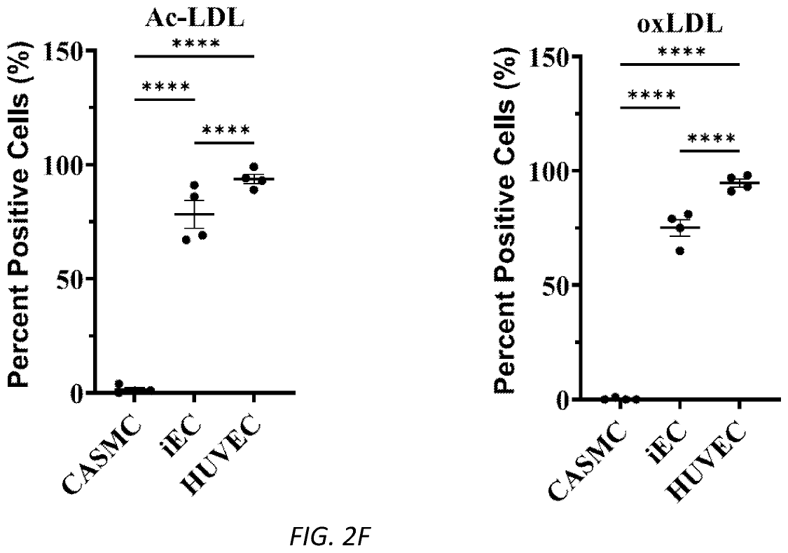

Confocal micrographs of these markers further demonstrated their transformed state as iECs exhibited appropriate endothelial localization patterns for CD31, VE-Cadherin, and KDR that resembled those of HUVECs and at significantly higher intensities than CASMCs (FIG. 2A). The expression levels of these EC markers in iECs were within the range of that found in other widely used primary EC lines including CAECs, HAECs, and HUVECs. In contrast, iECs exhibited reduced intensities of immunofluorescent signals for SMC proteins such as αSMA, compared to CASMCs (FIG. 2C, D). Because the expression or lack thereof are not true indicators of EC function, a brief functional investigation into the EC-like phenotype of iECs was evaluated by the uptake of acetylated low-density lipoprotein (Ac-LDL) and oxidized low-density lipoprotein (oxLDL). We analyzed two different lipids as the modified-LDL uptake function of ECs is mosaic when compared to many arterial cells, including SMCs. Confocal micrographs of cells treated with DiI-labeled Ac-LDL and oxLDL indicated that iECs uptake these lipids in a manner that is consistent with HUVECs with no signal observed in CASMCs (FIG. 2E, F).

### Example 3. Transcriptomic Profiling of iECs, HUVECs, and CASMCs

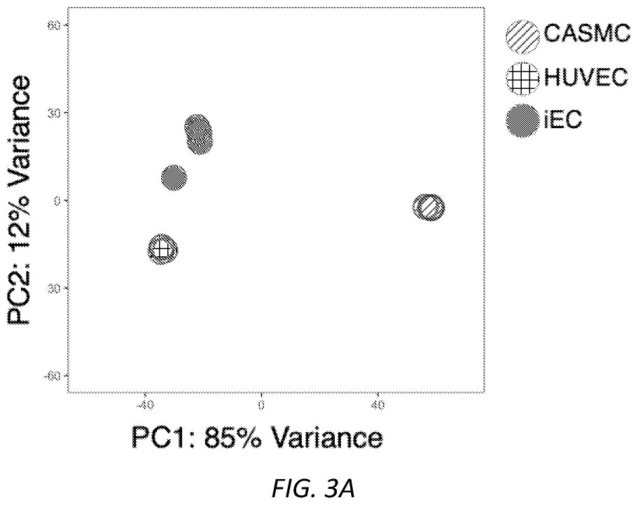

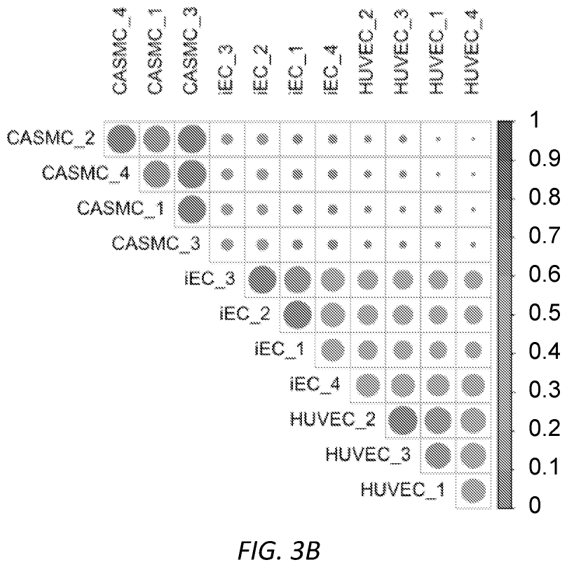

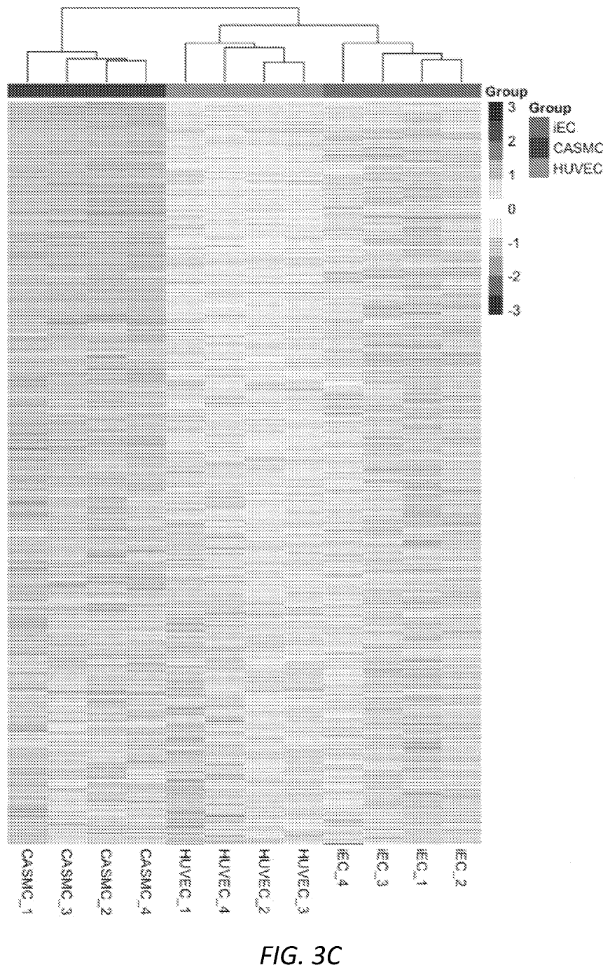

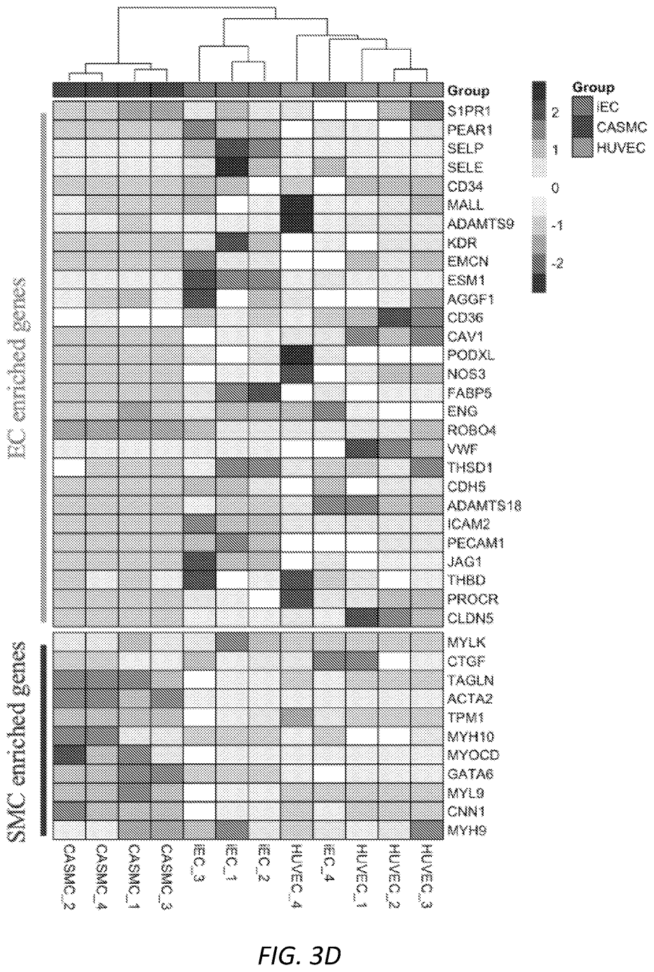

In order to obtain a comprehensive portrait of the relative transcriptomic similarity of iECs to HUVECs, or CASMCs, we conducted genome-wide RNA-seq. Principal component analysis (PCA) of the whole transcriptome finds that iECs cluster more closely to HUVECs than to CASMCs with a higher similarity index to HUVECs compared to CASMCs based on Euclidian distance (FIGS. 3A and B, S7). Further analysis of unsupervised hierarchical heatmap clustering of sample groups finds that whole transcriptome profiling results in clustering between iECs and HUVECs, followed more distantly by CASMCs (FIG. 3C), Narrowing the genes of interest to endothelial-specific transcripts based on a curated gene set (26), such as vWF, CDH5, CD31, NOS3, and others, followed by a second round of heatmap clustering demonstrate that iEC transcriptome profiles cluster closer to HUVECs (FIG. 3D). A similar comparison of selected SMC genes demonstrated that iECs maintain hierarchical clustering proximity to HUVECs under these parameters (FIG. 3D).

### Example 4. Pathway Enrichment Analysis of Endothelial-Specific Gene Networks in iECs

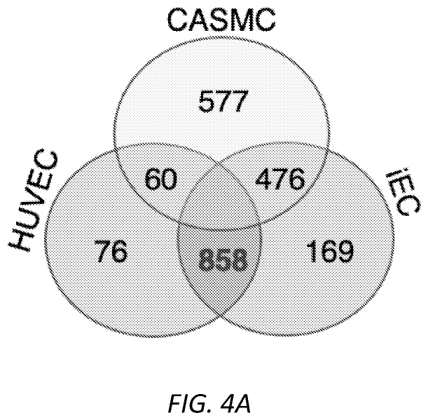

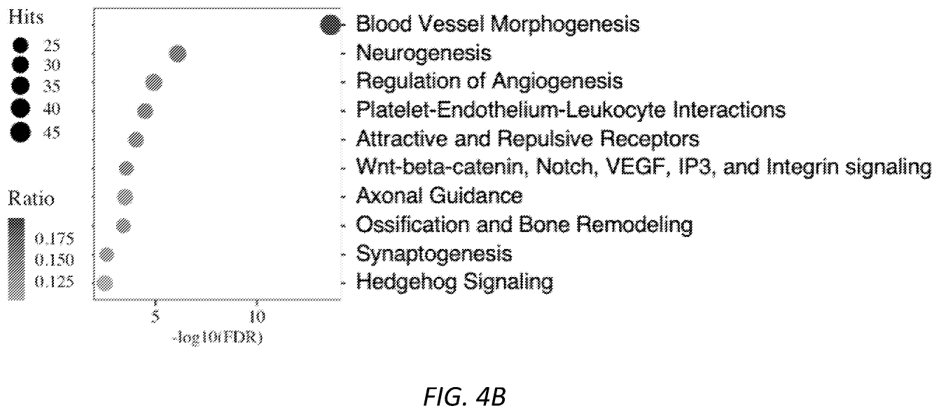

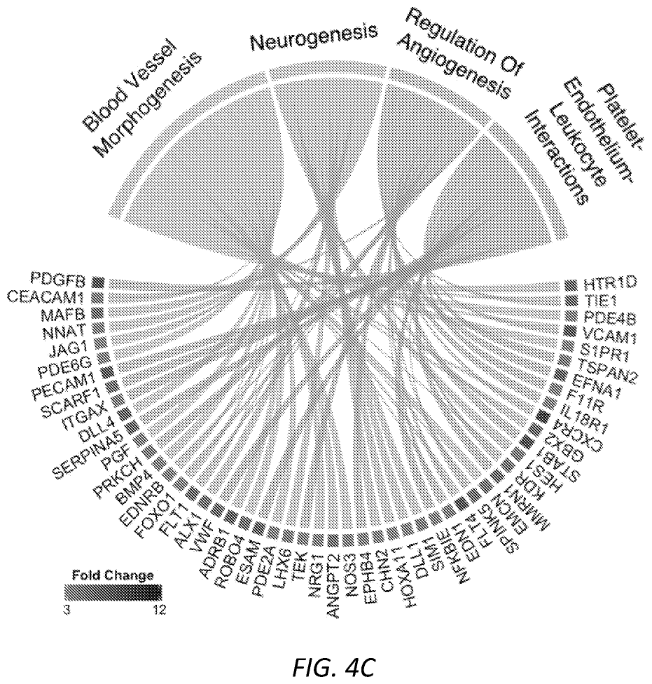

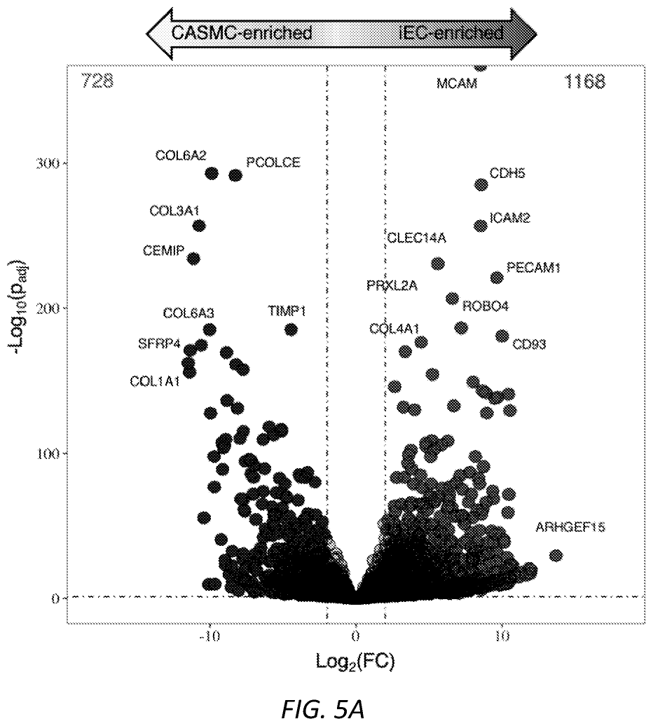

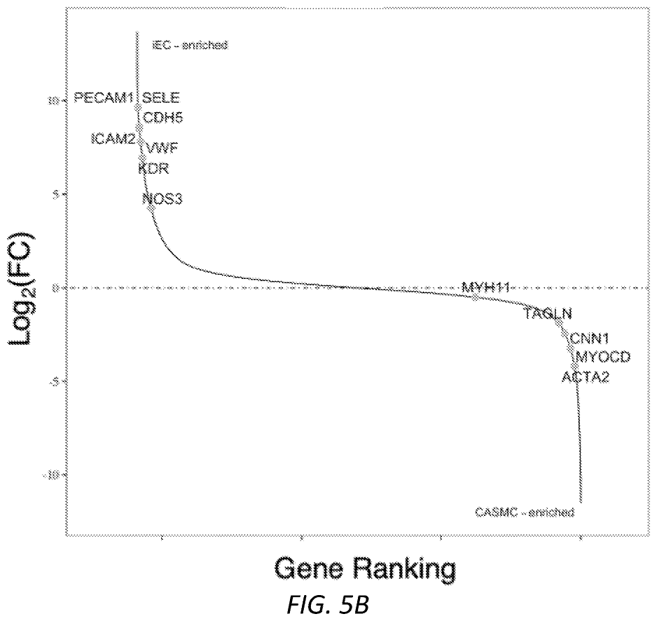

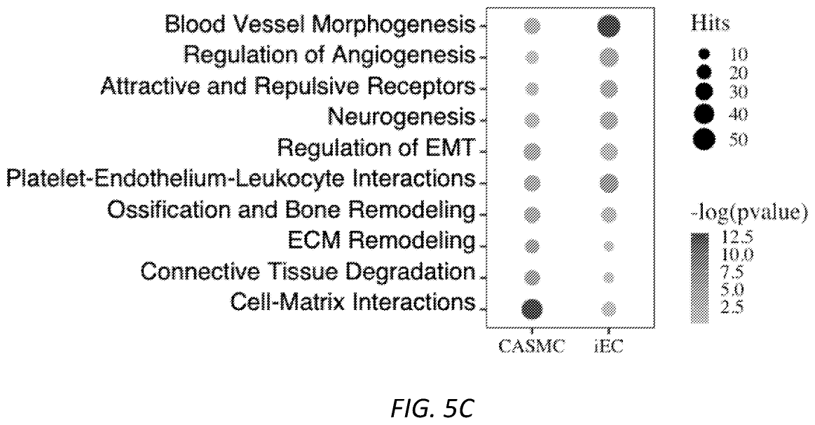

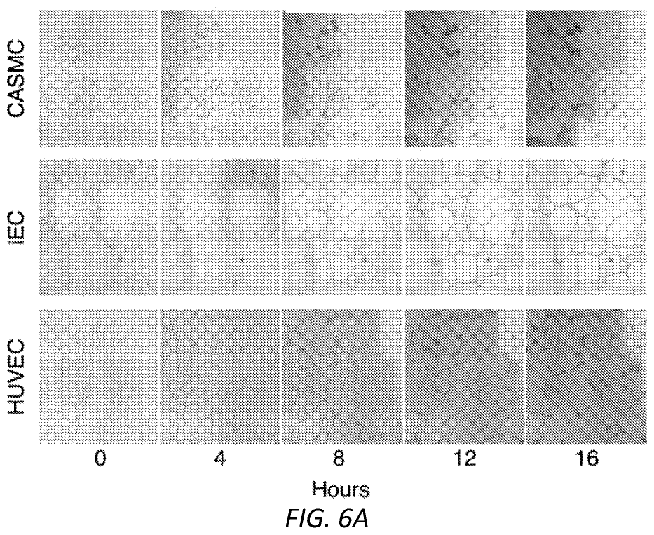

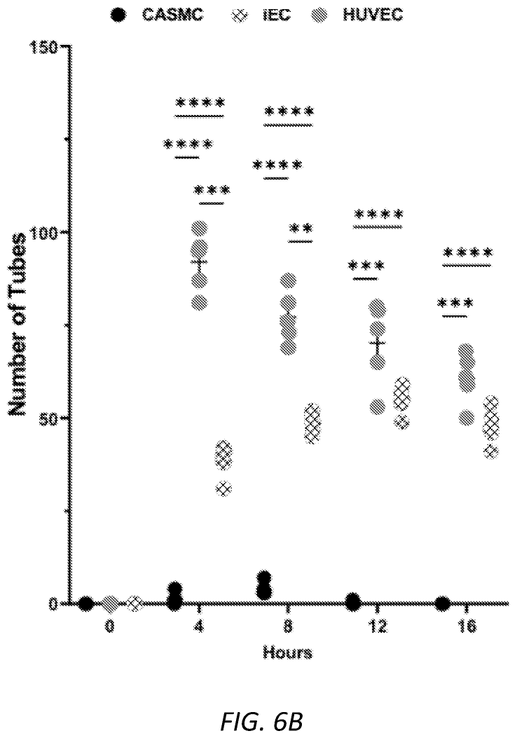

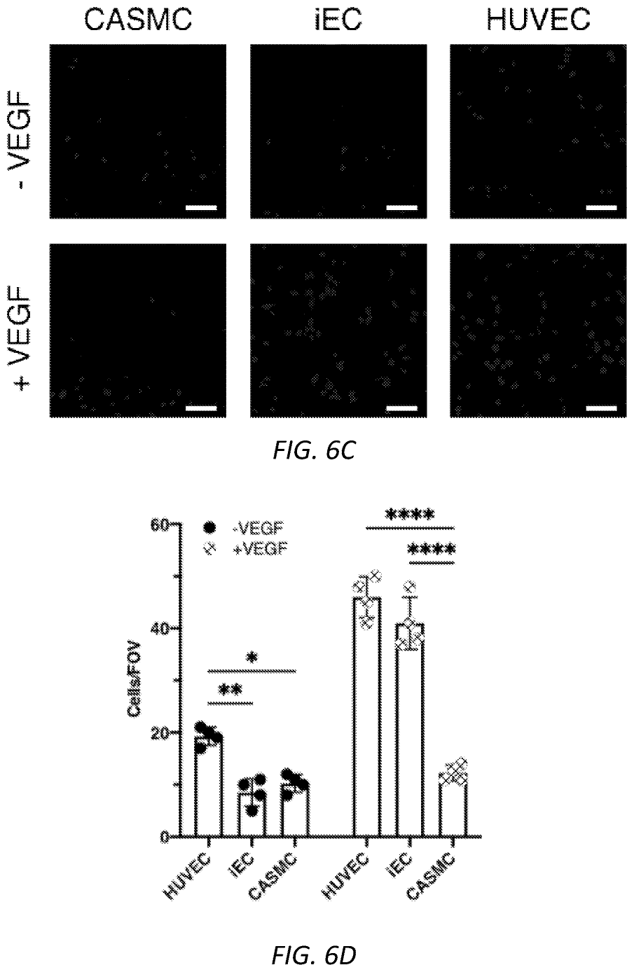

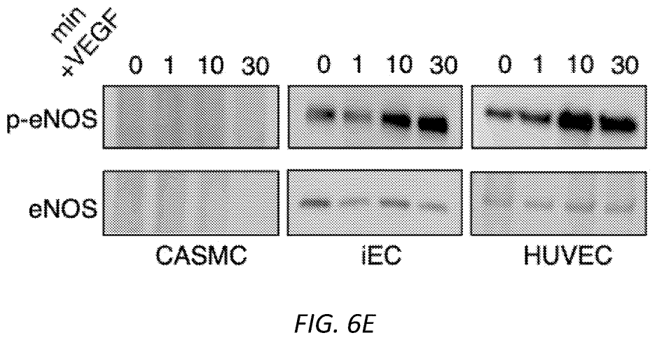

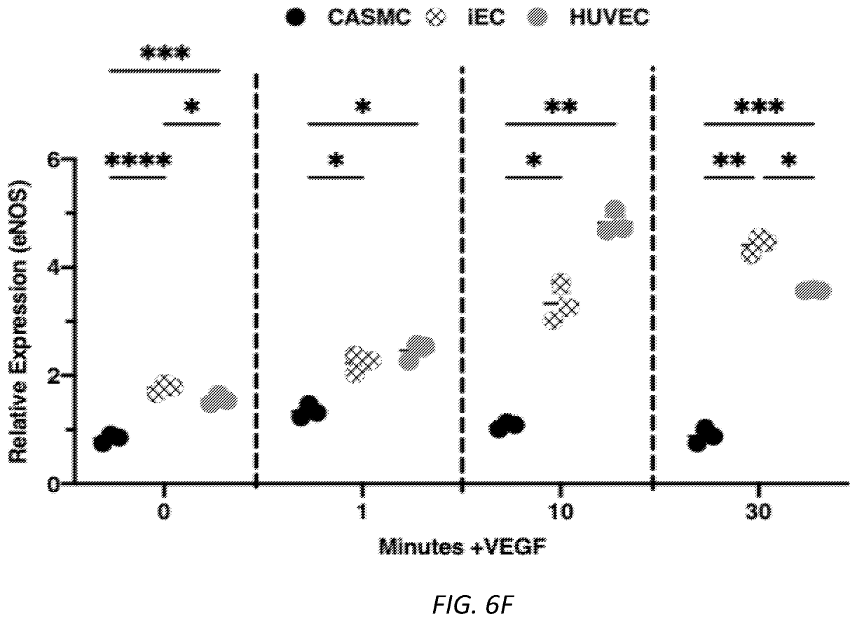

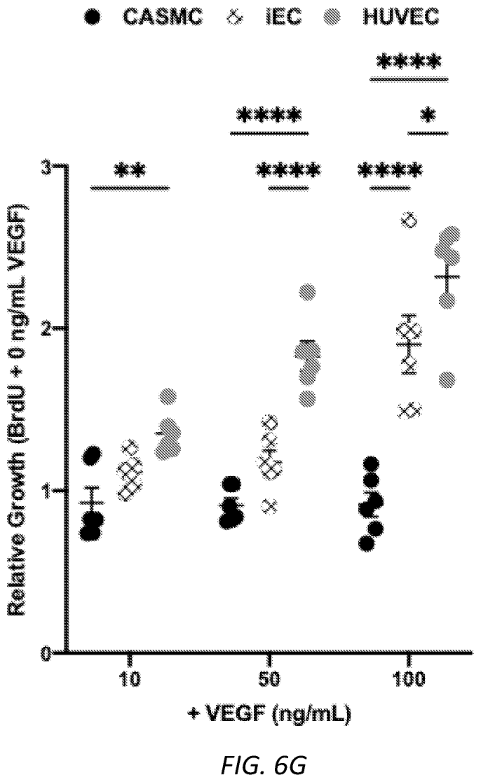

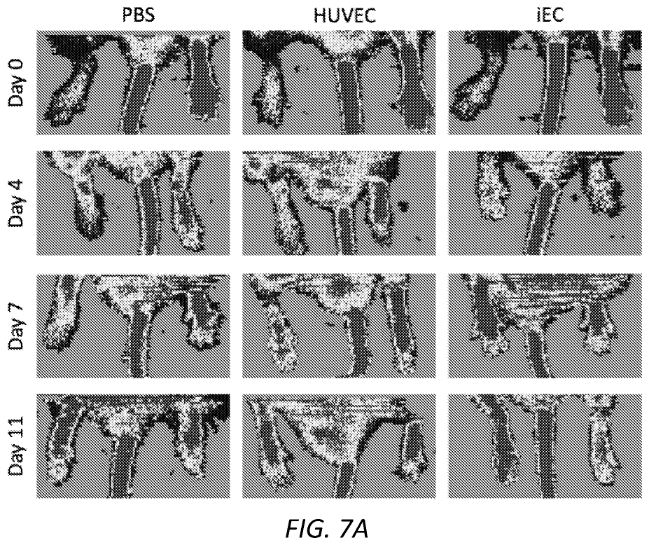

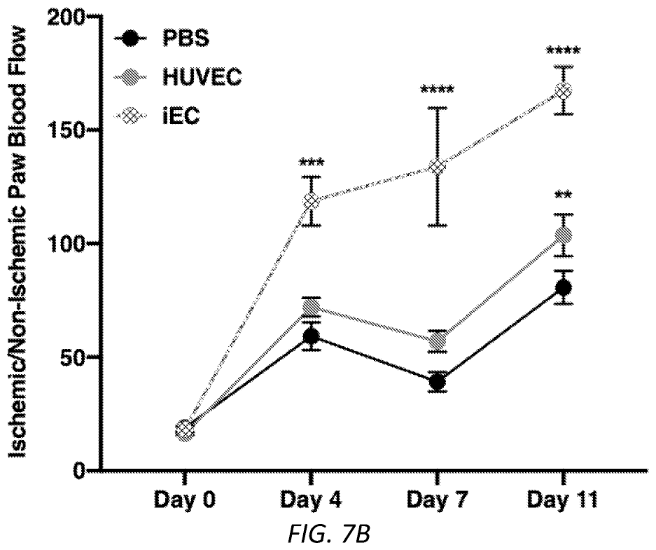

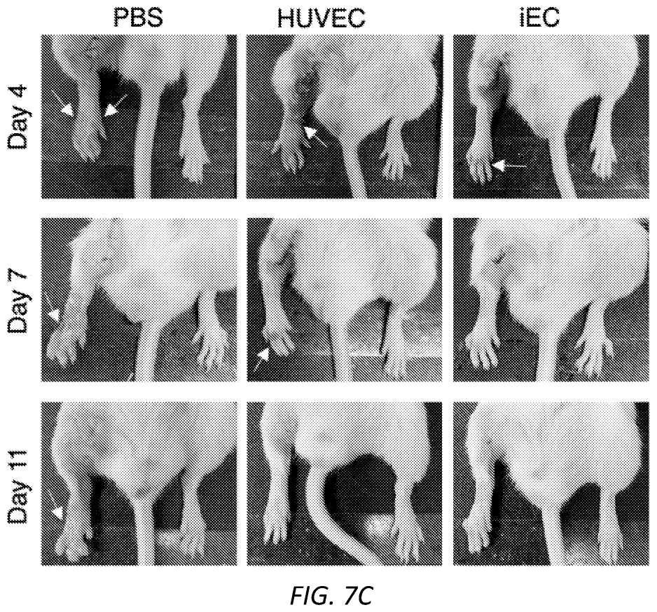

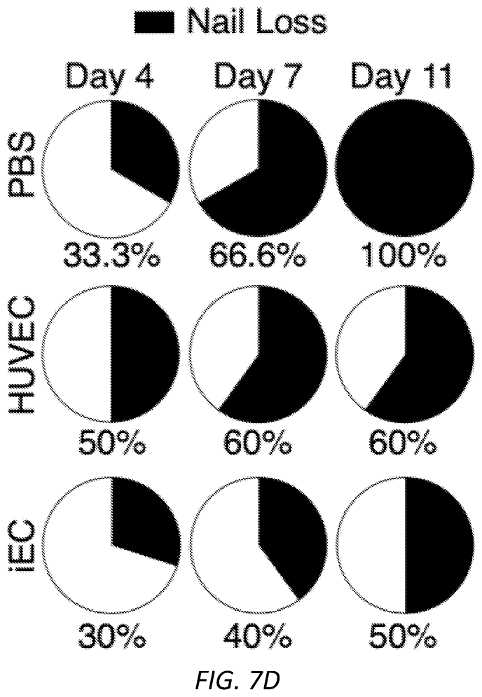

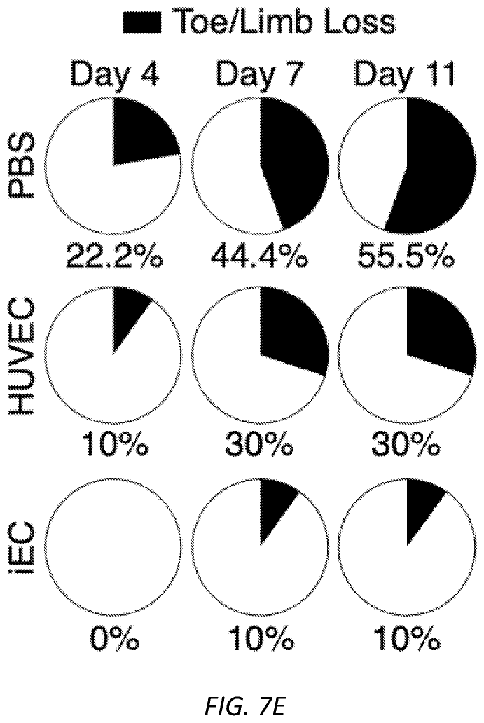

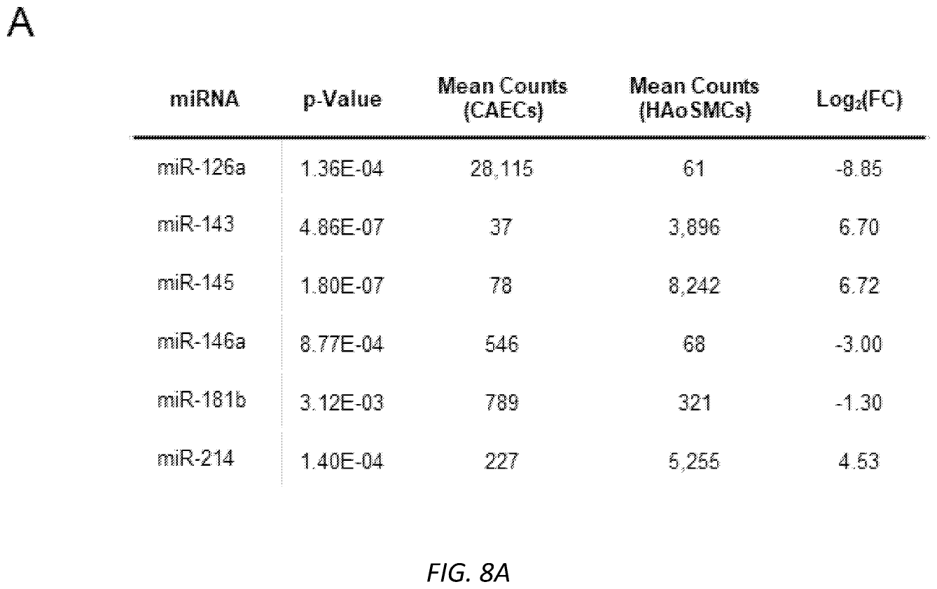

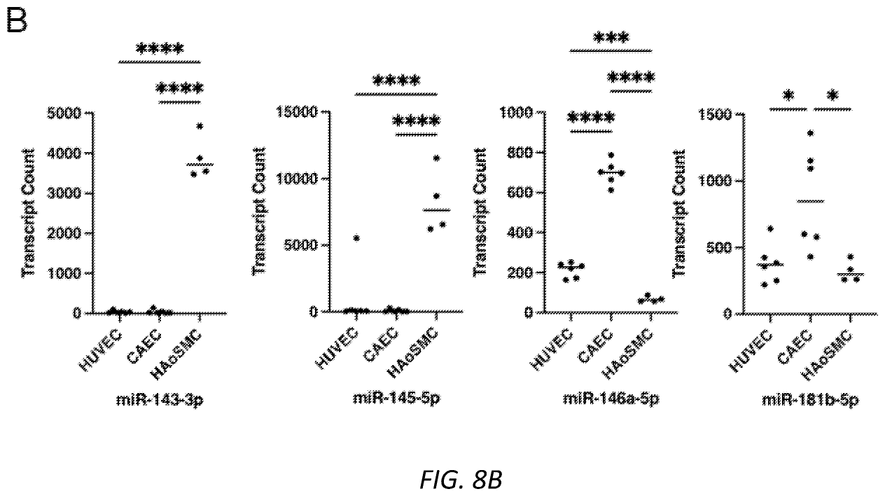

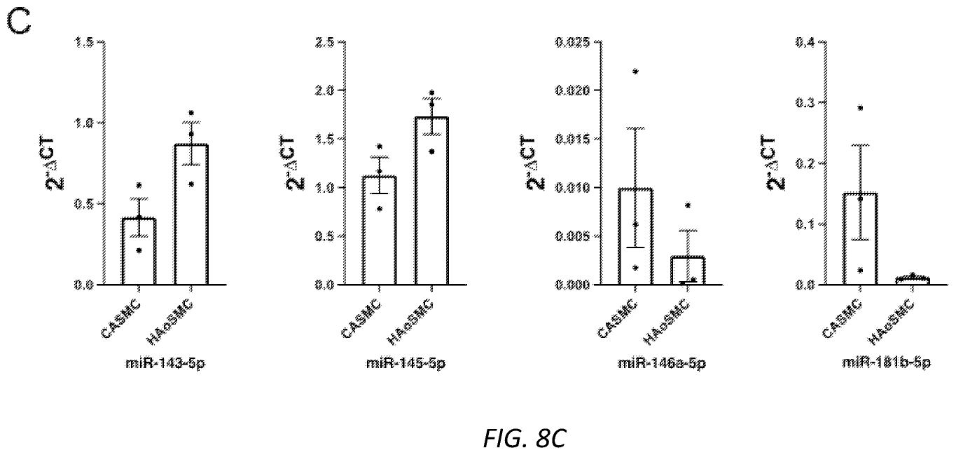

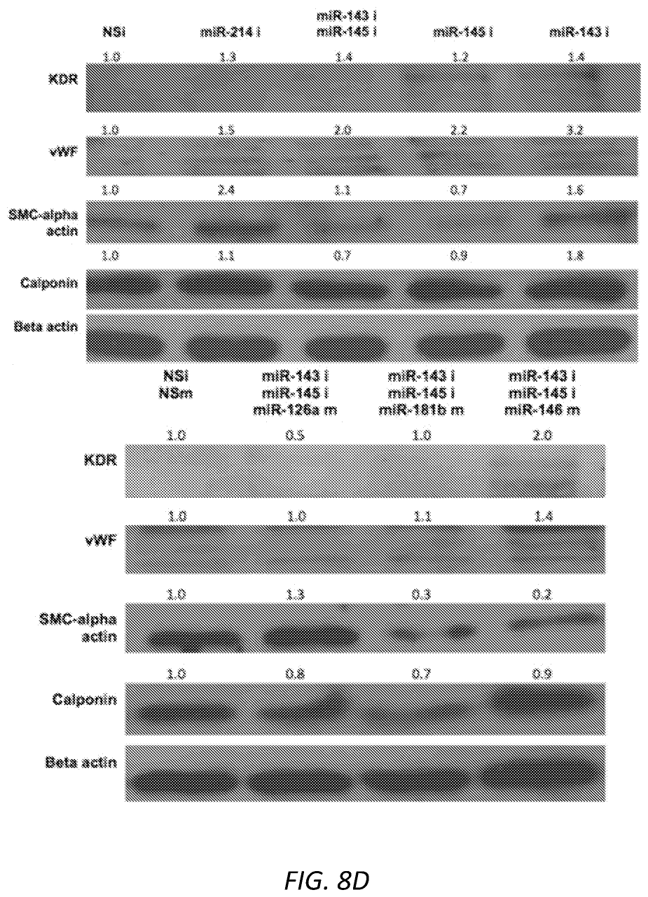

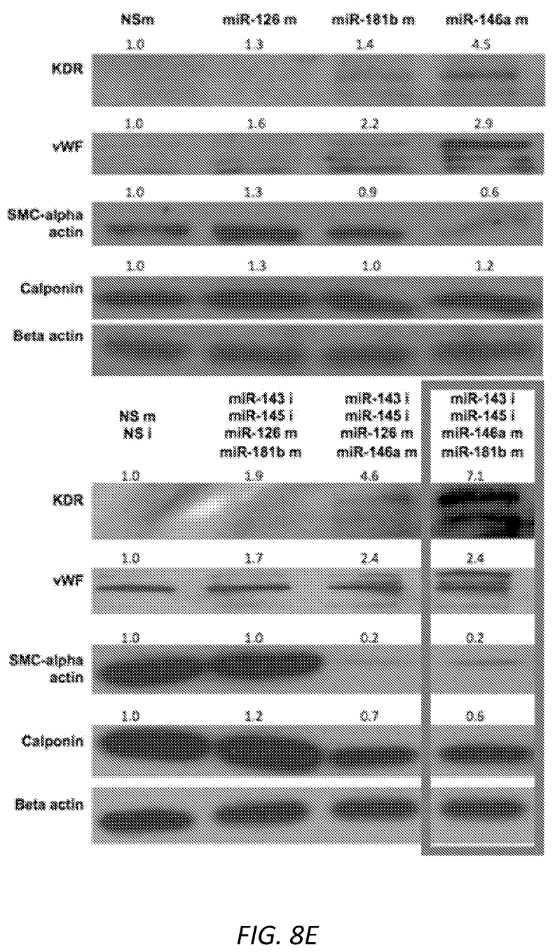

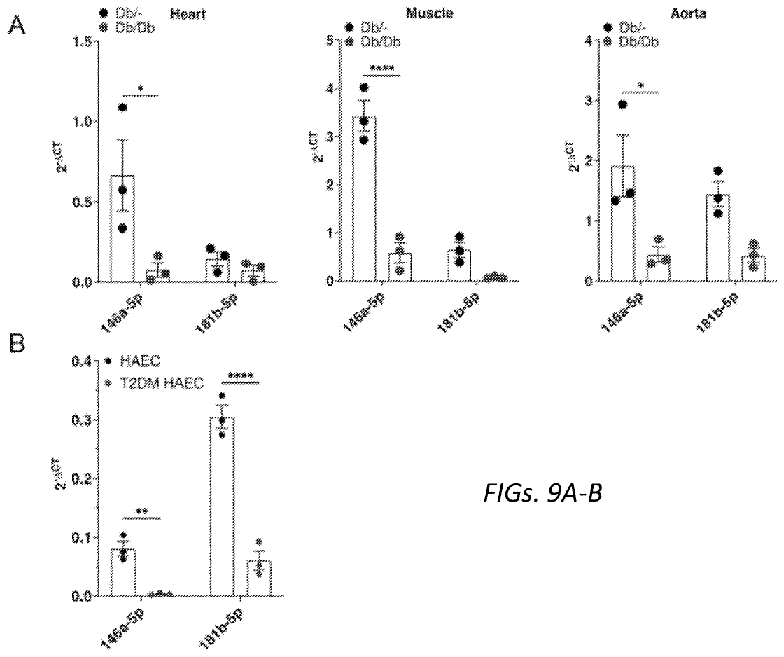

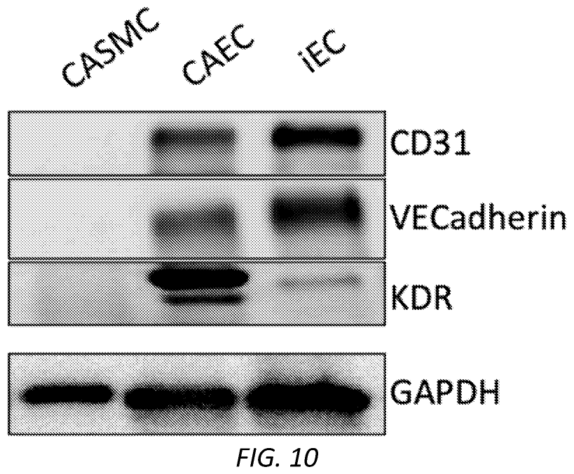

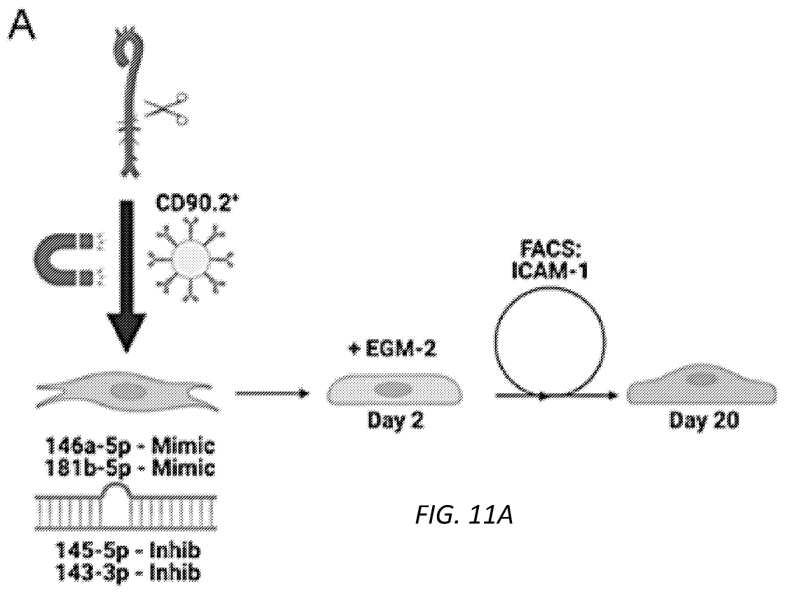

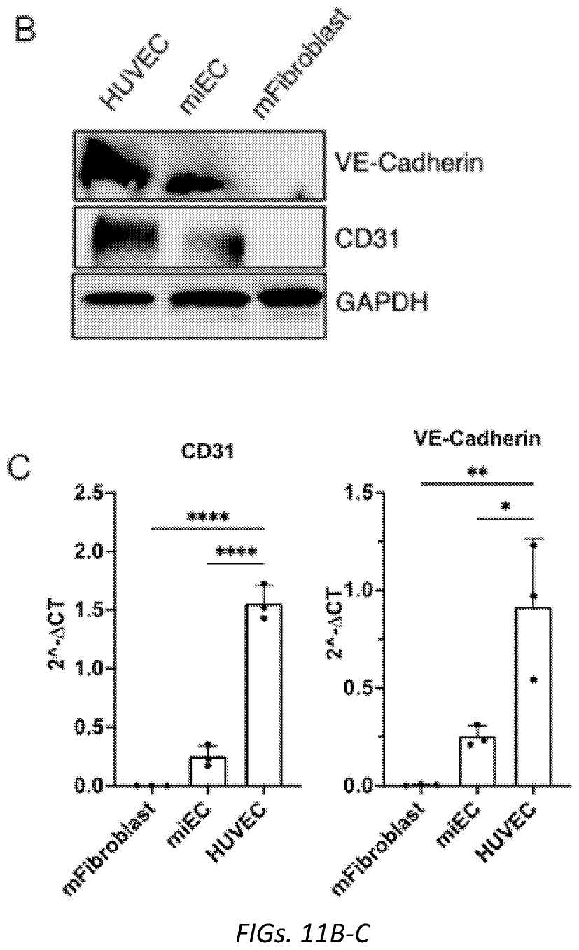

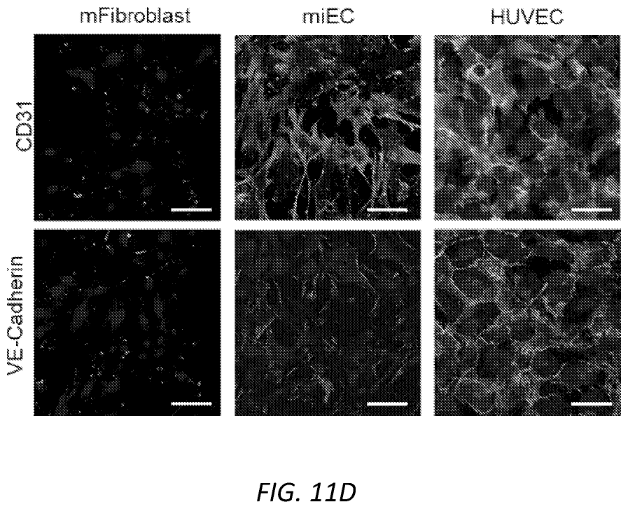

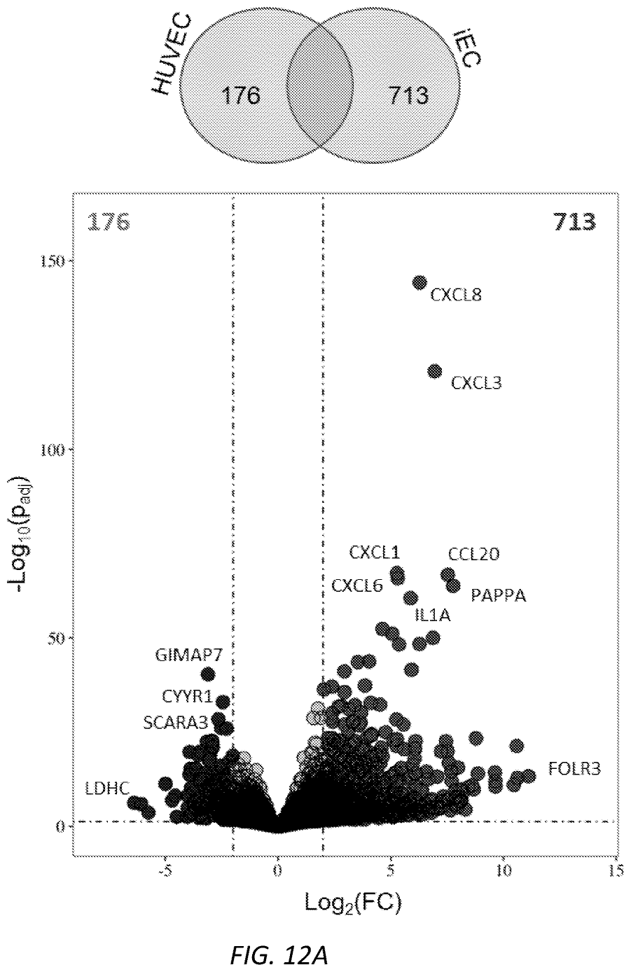

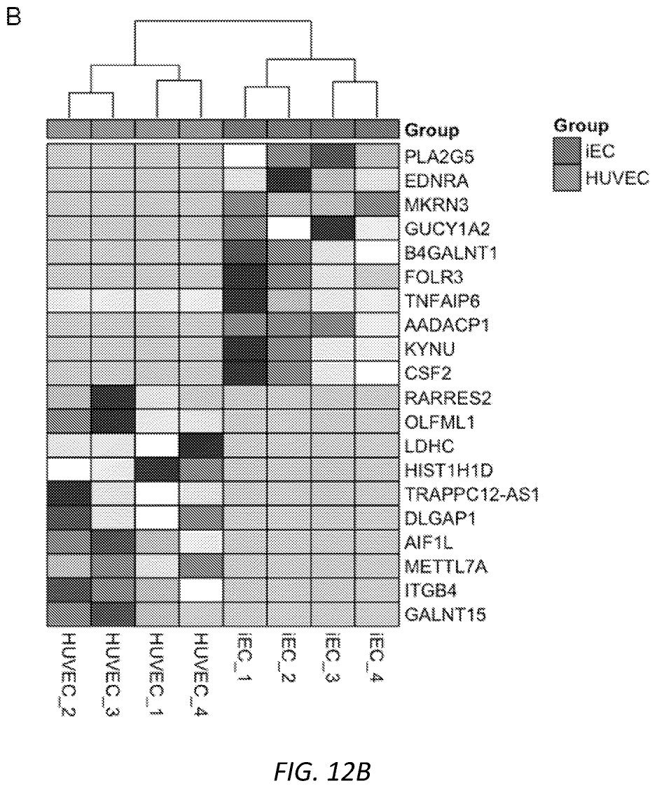

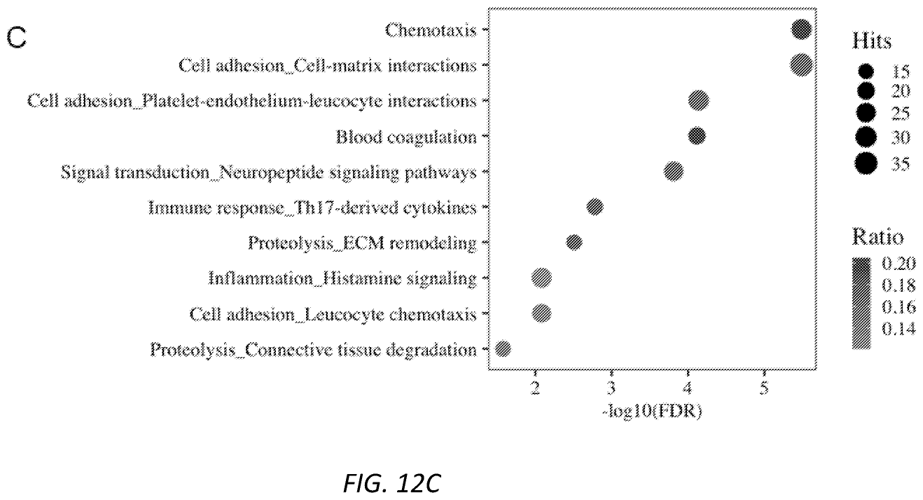

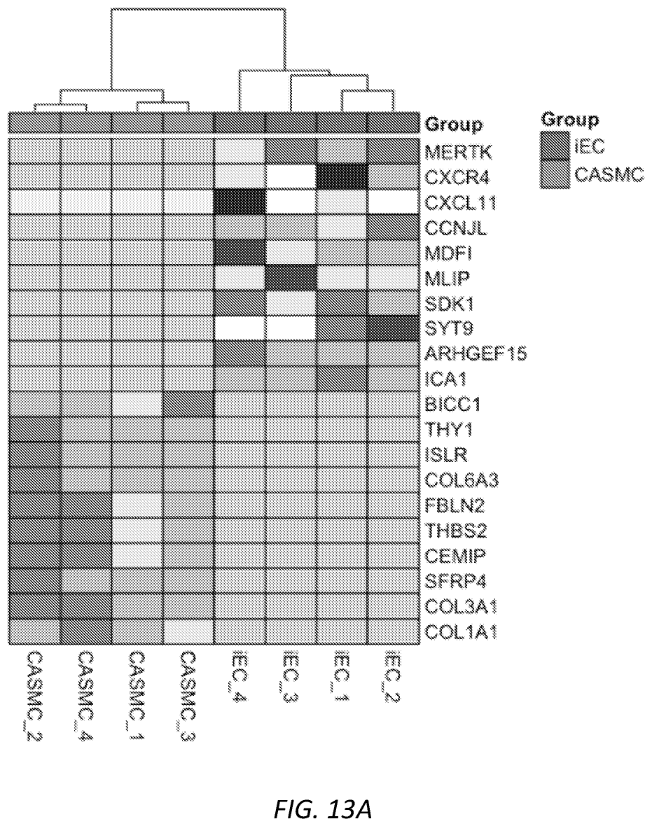

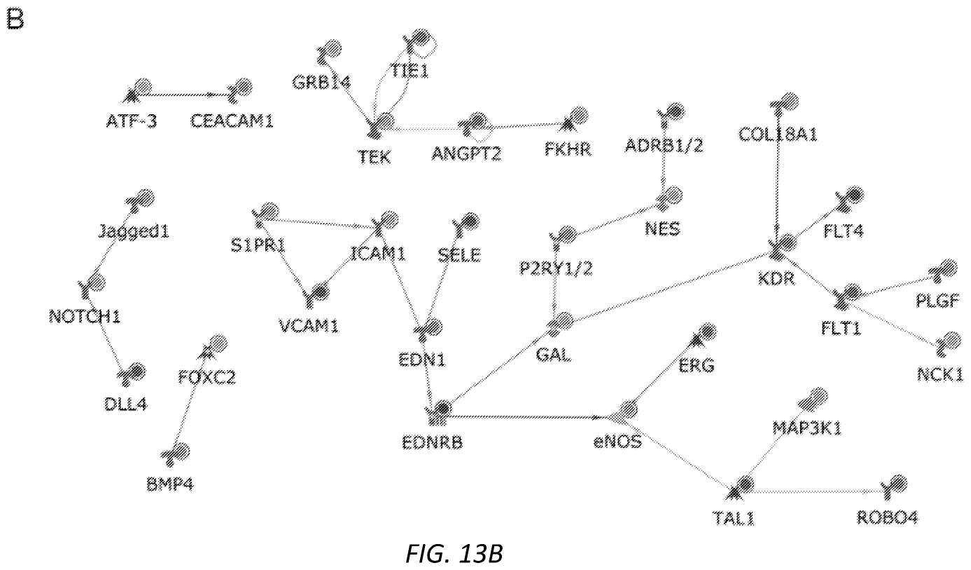

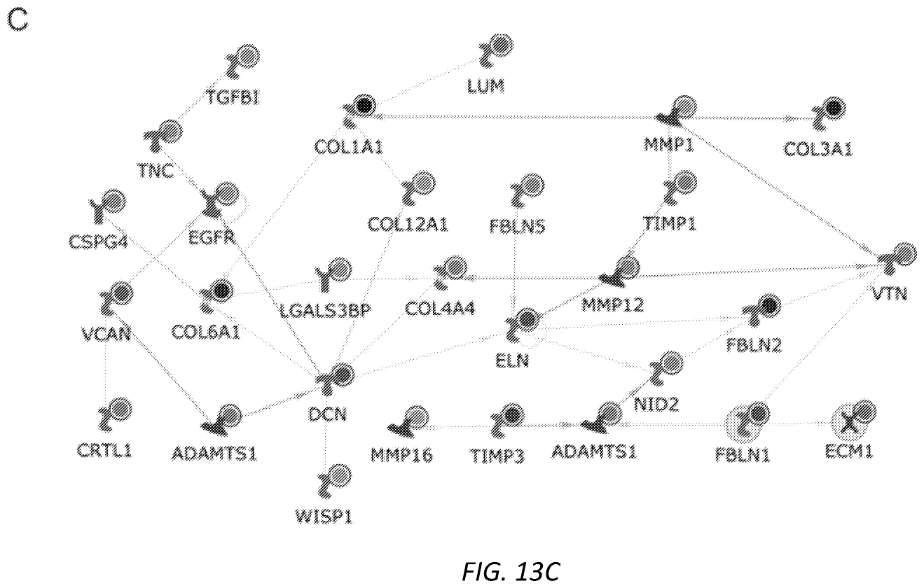

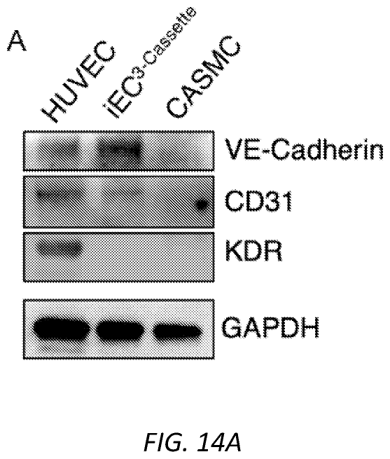

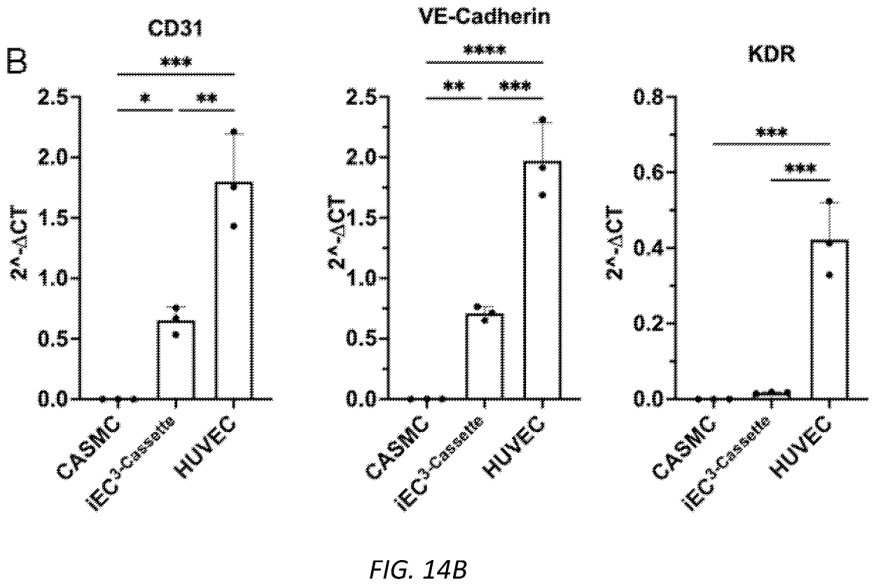

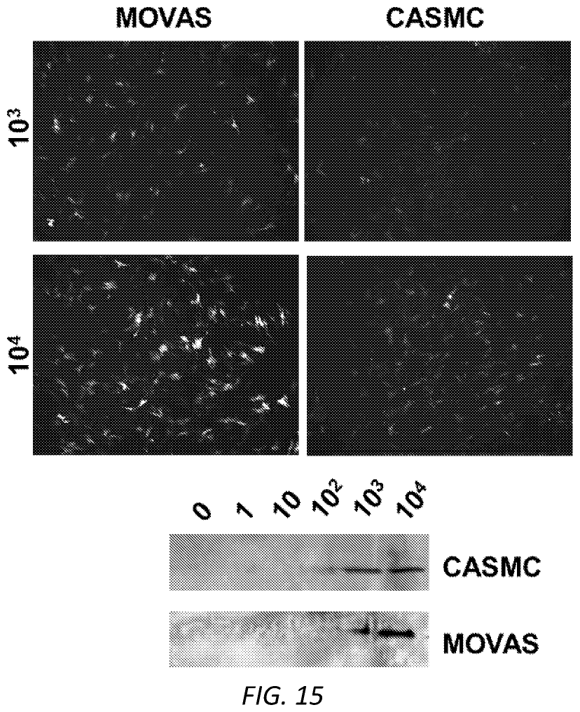

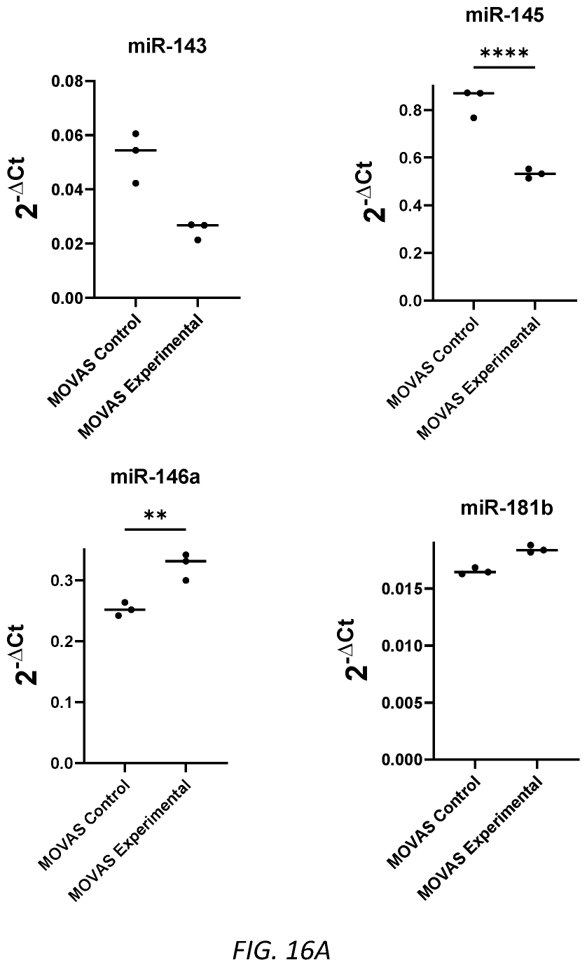

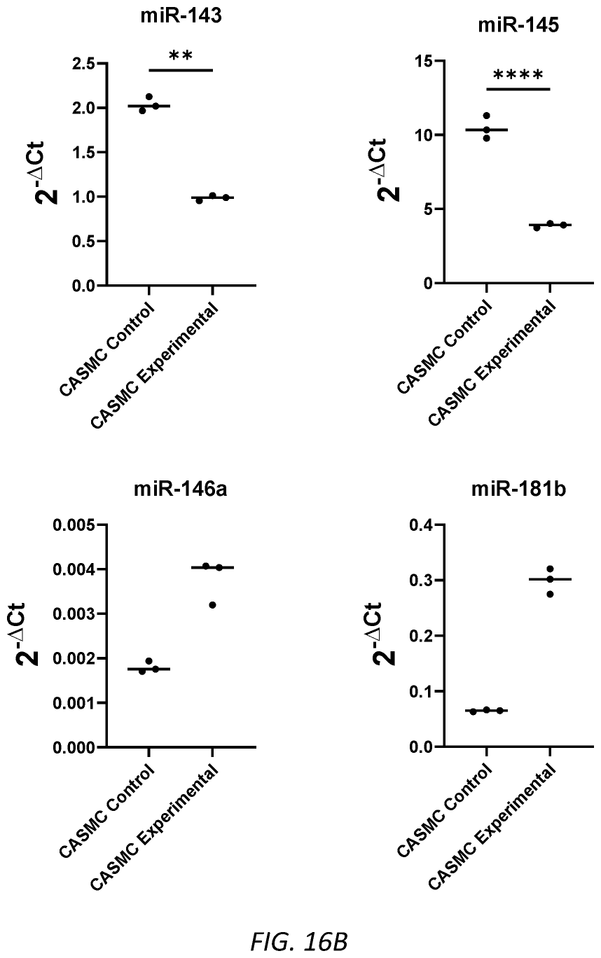

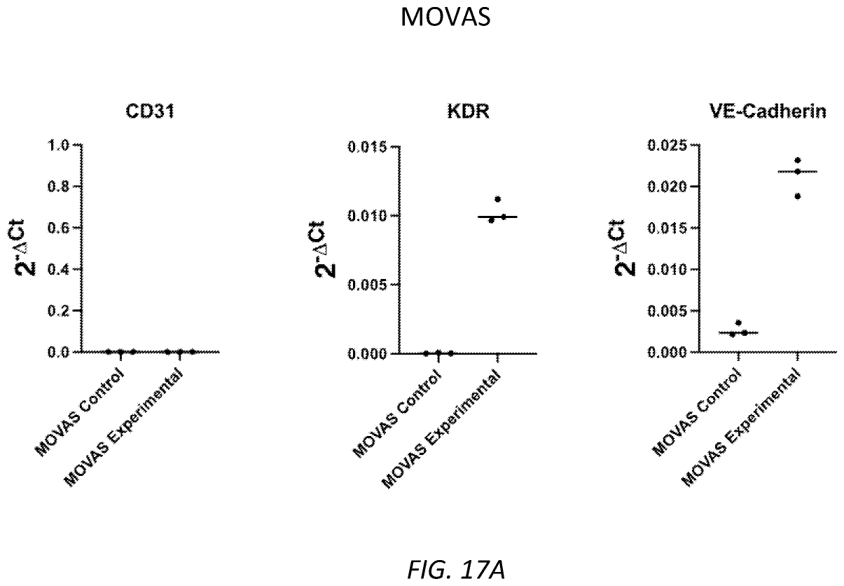

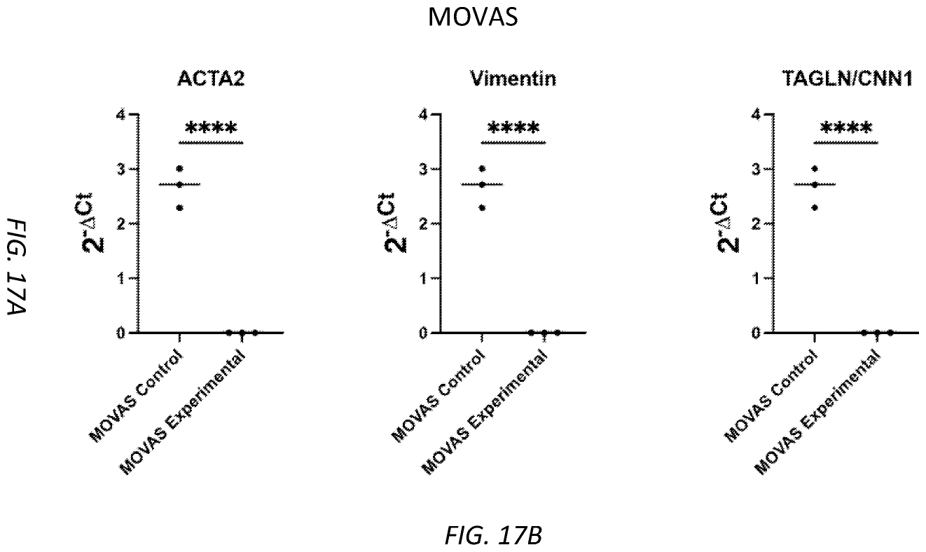

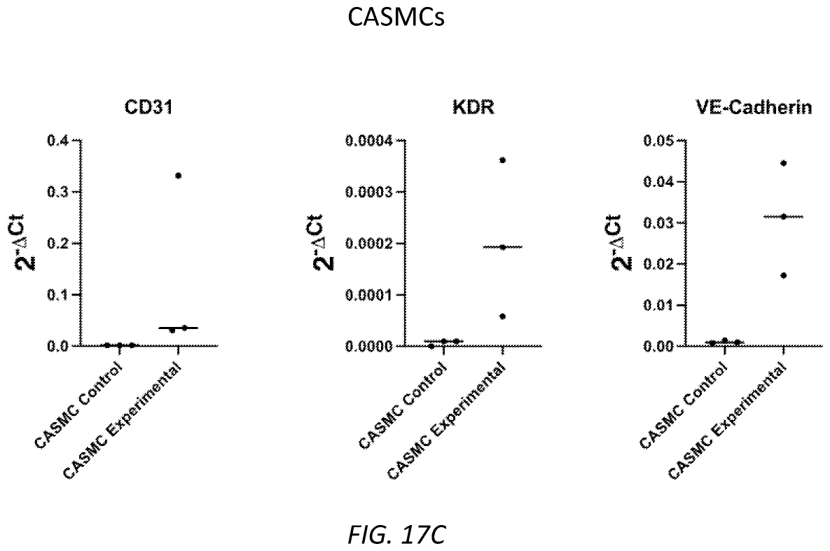

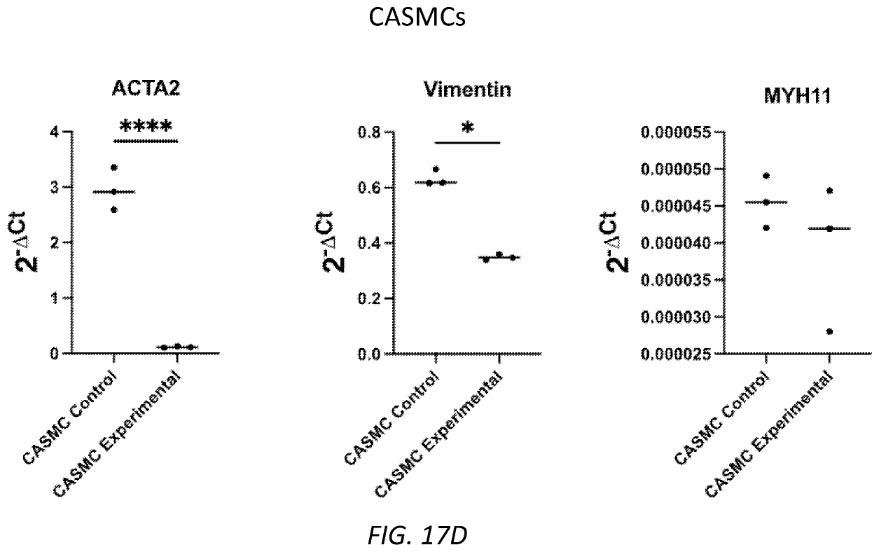

Utilizing this RNA-seq dataset, we sought to determine which processes and pathways were similarly regulated between iECs and HUVECs but did not overlap with gene networks within CASMCs in order to isolate possible endothelial pathways (FIG. 4A). Through this gene set enrichment analysis (GSEA), identification of the top gene networks were organized by log₁₀(False Discovery Rate (FDR)) and the number of common differentially expressed genes are shown within each network (log fold change>2.0, p_{adj}<0.05, Metacore process networks). This analysis revealed that the overlapping differentially regulated gene sets between iECs and HUVECs compared to CASMCs were linked to EC function (FIG. 4B). These gene networks included blood vessel morphogenesis, regulation of angiogenesis, platelet-endothelium-leukocyte interactions, and Wnt-beta-catenin, vascular endothelial growth factor (VEGF), inositol trisphosphate 3 (IP3), and integrin signaling; pathways. To further visualize the overlapping gene networks and their specific gene components, a chord diagram of the top 4 process networks was generated and demonstrates affected overlapping genes as belonging to canonical endothelial cell function pathways (FIG. 4C). A GSEA of computationally predicted upregulated and downregulated targets of the 4-miRNA combination found overlap of several similar gene pathways: connective tissue degradation, ECM remodeling, and regulation of epithelial-to-mesenchymal transition were among the top for miR-181b-5p and miR-146 mimics, whereas pathways enriched for anti-apoptotic pathways related to PI3K/AKT and JAK-STAT. and actin and intermediate filaments were represented by miR-143/miR-145 anti-miRs. A comparison data set and GSEA was generated to identify differentially regulated genes between iECs and HUVECs, finding only a few endothelial cell specific genes to be differentially expressed (FIGS. 12A, B, C).

### Example 5. Transcriptome Divergence Between iECs and CASMCs

Knowing that iECs were transcriptionally comparable to 1-HUVECs, our next analysis assessed the degree of dissimilarities between the iECs and CASMCs. These data found iECs to contain 1168 differentially expressed genes compared to CASMCs (log₂ fold change>2.0, p_{adj}<0.05), with endothelial genes such as CDH15, CD31, and ICAM2 to be among the most highly upregulated differentially expressed transcripts (FIG. 5A). In comparison, there were 728 genes differentially expressed transcripts in CASMCs including many enriched for the collagen synthesis pathways (FIG. 13A). Further organization of expression profiles alongside gene ranking determined that iEC transcripts are more abundant in EC. markers, such as CD31, CDH5, vWF, and KDR, and cluster several-fold away from SMC genes (FIG. 5B). Smooth muscle transcripts by comparison demonstrated a downregulation in markers for ACTA2, transgelin (TAGLN), and MYH11 in iECs. GSEA also identified process networks representative of EC function such as blood vessel morphogenesis, regulation of angiogenesis, and platelet-endothelium-leukocyte interactions further confirming a significant upregulation of endothelial-associated gene networks in iECs (FIG. 5C, 13B). Conversely, process network analysis for CASMCs showed an enrichment of SMC related networks such as ECM remodeling, connective tissue degradation, and cell-matrix interactions that is absent in iECs (FIG. 13C).

### Example 6. Functional Capacity of iECs

To couple the transcriptional, translational, and phenotypic changes in iECs to their functional role as ECs, the last set of analyses focused on their ability to function as ECs. A Matrigel™ tube formation assay confirmed that iECs exhibit multicellular endothelial-like behavior as iECs and formed matrix networks when compared to CASMCs (FIG. 6A). While HUVECs underwent quicker tube formation, iECs were not significantly different from HUVECs at later time points, demonstrating similar network kinetics over the course of the assay (FIG. 6B). Next, we sought to investigate iEC chemotactic responses to VEGF signaling and assessed the migratory capacity of iECs in response to VEGF stimulation compared to HUVECs and CASMCs. While all cell types exhibited low levels of migration under basal conditions, HUVECs and iECs migrated approximately 2.5-fold as much with the addition of VEGF; in contrast, CASMC migration was not affected by VEGF (FIG. 6C). Because these data indicated that iECs respond to VEGF stimulation and our RNA-seq data identified that NOS3 (eNOS) (FIG. 3, 5) was upregulated in iECs, we evaluated if VEGF stimulation resulted in phosphorylation of eNOS. Protein lysates of samples stimulated for 0, 1, 10, and 30 minutes with VEGF were collected and Western blots were performed for CASMCs, iECs, and HUVECs (FIG. 6D). A characteristic time-dependent phosphorylation signal in eNOS verified that iECs responded to VEGF and followed a similar induction achieved in HUVECs but not in CASMCs. Lastly, BrdU labeling of C SMCs, iECs, and HUVECs was carried out to determine whether iEC growth was affected by the supplementation of VEGF. These data revealed a dose-dependent growth effect of VEGF on iECs that tracked with HUVECs, while having had no significant effect on CASMCs (FIG. 6E).

### Example 7. Hindlimb Ischemia Recovery is Improved with the Addition of iECs

To validate if iEC similarity to ECs in vitro was indicative of a potential functional capacity in vivo, a mouse hindlimb blood flow recovery approach was utilized. The femoral artery of the right hindlimb in NOD SCID mice was ligated and cauterized to ablate blood flow. After 24 hours, PBS, iECs, or HUVECs were injected into the tail vein and blood flow was monitored via laser Doppler imaging (FIG. 7A). While Doppler images of the hindlimb demonstrate a recovery of blood flow over time in all three conditions over 14 days, iEC-injected mice exhibit a quicker reperfusion of the limb than those of PBS- or HUVEC-inject mice at days 4 (by 206% and 161%, respectively), 7 (by 344% and 235%, respectively), and 11 (by 270% and 142%. respectively) (FIG. 7B) it is also important to note that while PBS and HUVEC groups exhibited no differences prior to 7 days post-surgery, HUVEC-injected mice trended towards quicker recover and exhibited significantly faster recovery by 11 days (by 185%). Beyond Doppler imaging, visual observations of the limbs further demonstrated the differences between each condition as iEC-injected mice exhibited fewer visual signs of tissue damage and necrosis when compared to PBS or HUVEC conditions (FIG. 7C). The differences between PBS- and HUVEC-injected mice displayed similar trends to Doppler imaging. Quantification of nail, toe, and limb amputation further confirmed this observation as PBS and HUVEC-injected mice were more likely to lose nails and toes in comparison to the iEC group (FIG. 7D, E). While no engraftment of iECs or HUVECs was observed at this experimental endpoint, the iEC-injected mice exhibited higher levels of eNOS within the endothelium of skeletal muscles, providing a plausible mechanism for how iECs may mediate their proangiogenic effects (FIGS. 14A, B). The angiogenic paracrine effects of promoting more blood flow and increasing angiogenic markers (e.g. eNOS) was quite apparent. These data suggest that iECs are more potent than traditional ECs at facilitating blood flow and limb ischemia recovery.

### Example 8. AAV for Expression of Four miRNA Cassette

A miRNA expression construct including sequences encoding two miRNA mimics, of miR-146a and miR-181b, and two copies of a TuD RNA inhibitor of miR-143 and miR-145, was developed. The construct was inserted into AAV9 or AAV2 and expression of all of the above was driven by a single SM22a promoter, with a WPRE element.

The sequences of the mimics used comprise:

miR-146a sequence (5′-3′)
(SEQ ID NO: 39)
CCGAUGUGUAUCCUCAGCUUUGAGAACUGAAUUCCAUGGGUUGUGUC
AGUGUCAGACCUCUGAAAUUCAGUUCUUCAGCUGGGAUAUCUCUGUC
AUCGU
miR-181b-2 sequence (5′-3′)
(SEQ ID NO: 40)
CUGAUGGCUGCACUCAACAUUCAUUGCUGUCGGUGGGUUUGAGUCU
GAAUCAACUCACUGAUCAAUGAAUGCAAACUGCGGACCAAACA

The first portion of the cassette was generated using the following sequences:

250 nucleotide flanking sequence w/ miR-146a
(5′-3′):
(SEQ ID NO: 41)
TTTAGTAGAGACAAATTCTCCATGTTGCCCAGGCTAGTCCTGAACTCCTGG
GCTCAAGAGATCCACCCACATCAGCCTTCCAGACTGCTGGCCTGGTCTCCT
CCAGATGTTTATAACTCATGAGTGCCAGGACTAGACCTGGTACTAGGAAG
CAGCTGCATTGGATTTACCAGGCTTTTCACTCTTGTATTTTACAGGGCTGG
GACAGGCCTGGACTGCAAGGAGGGGTCTTTGCACCATCTCTGAAAAGCCG
ATGTGTATCCTCAGCTTTGAGAACTGAATTCCATGGGTTGTGTCAGTG
TCAGACCTCTGAAATTCAGTTCTTCAGCTGGGATATCTCTGTCATCGT
GGGCTTGAGGACCTGGAGAGAGTAGATCCTGAAGAACTTTTTCAGTCTGC
TGAAGAGCTTGGAAGACTGGAGACAGAAGGCAGAGTCTCAGGCTCTGAA
GGTATAAGGAGTGTGAGTTCCTGTGAGAAACACTCATTTGATTGTGAAAA
GACTTGAATTCTATGCTAAGCAGGGTTCCAAGTAGCTAAATGAATGATCT
CAGCAAGTCTCTCTTGCTGCTGCTGCTACTCGTTTACATTTATTGATTACT
250 nucleotide flanking sequence w/ miR-181b-2
(5′-3′):
(SEQ ID NO: 42)
GCCTGTGTGGCGCAGTGCTACCTGTGAGGTTCTCCAAGCACTCCTTCCTTC
TCTGAACCACAGCTTCCTCATCTGCAATAACCTCCCAGCTCCAATGTCAAT
GTTATGGACACCTGTGTGGGCCCTCAATCATGCAGATGGCTGGTTACTAA
GGGAGAAGCCAGACACACAGACTTCAAAGAACTGAGATGGAAAAGAAGA
GCCAGGAGTCAGCCAGGGAGGGCAAAGGCAACCCCACCAACTGAAAACA
CTGATGGCTGCACTCAACATTCATTGCTGTCGGTGGGTTTGAGTCTGA
ATCAACTCACTGATCAATGAATGCAAACTGCGGACCAAACACCCAAAG
GAAGCAAAAGCATGTGCAGAGAGTCGCCTGAGTGCAGCAGCTACTGCTGT
GAGTGGGAGCTGCTACTATTTCCAAAACACTAGCTAAAGCTTTTCTTGTTC
TCCTTGGTTCTAACATGGAGTAAGTTCTCTAAATTCCTAGTACTTTTCTCC
TTTTTCAATAGTCTAGATTTAAGGAAATGATATTTTATAGGAAGAATTAAA
TACATATCTGGATATCACATCACATTTTAAAAATCAGTA

Combining these two, remove 25 hp from each, 1141 bp (5′-3′) miR-146a/miR-181b-2 miRNA mimic expression sequence:

(SEQ ID NO: 43)
TTTAGTAGAGACAAATTCTCCATGTTGCCCAGGCTAGTCCTGAACTCCTGG
GCTCAAGAGATCCACCCACATCAGCCTTCCAGACTGCTGGCCTGGTCTCCT
CCAGATGTTTATAACTCATGAGTGCCAGGACTAGACCTGGTACTAGGAAG
CAGCTGCATTGGATTTACCAGGCTTTTCACTCTTGTATTTTACAGGGCTGG
GACAGGCCTGGACTGCAAGGAGGGGTCTTTGCACCATCTCTGAAAAGCCG
ATGTGTATCCTCAGCTTTGAGAACTGAATTCCATGGGTTGTGTCAGTG
TCAGACCTCTGAAATTCAGTTCTTCAGCTGGGATATCTCTGTCATCGT
GGGCTTGAGGACCTGGAGAGAGTAGATCCTGAAGAACTTTTTCAGTCTGC
TGAAGAGCTTGGAAGACTGGAGACAGAAGGCAGAGTCTCAGGCTCTGAA
GGTATAAGGAGTGTGAGTTCCTGTGAGAAACACTCATTTGATTGTGAAAA
GACTTGAATTCTATGCTAAGCAGGGTTCCAAGTAGCTAAATGAATGATCT
CAGCAAGTCTCTCTTGCTGCTGCTGCTATGAGGTTCTCCAAGCACTCCTTC
CTTCTCTGAACCACAGCTTCCTCATCTGCAATAACCTCCCAGCTCCAATGT
CAATGTTATGGACACCTGTGTGGGCCCTCAATCATGCAGATGGCTGGTTAC
TAAGGGAGAAGCCAGACACACAGACTTCAAAGAACTGAGATGGAAAAGA
AGAGCCAGGAGTCAGCCAGGGAGGGCAAAGGCAACCCCACCAACTGAAA
ACACTGATGGCTGCACTCAACATTCATTGCTGTCGGTGGGTTTGAGTC
TGAATCAACTCACTGATCAATGAATGCAAACTGCGGACCAAACAACCC
AAAGGAAGCAAAAGCATGTGCAGAGAGTCGCCTGAGTGCAGCAGCTACT
GCTGTGAGTGGGAGCTGCTACTATTTCCAAAACACTAGCTAAAGCTTTTCT
TGTTCTCCTTGGTTCTAACATGGAGTAAGTTCTCTAAATTCCTAGTACTTT
TCTCCTTTTTCAATAGTCTAGATTTAAGGAAATGATATTTTATAGGAAGAA
TTAAATACATATCTGGATATCACATCACATTTTAAAAATCAGTA

The inhibitors used were as follows (5-3′):

miR-145 sponge:
(SEQ ID NO: 44)
AGGGATTCCTTTTAAACTGGAC
miR-143 sponge:
(SEQ ID NO: 45)
GAGCTACAGTGCTTCATCTGA

A tough decoy approach was also used in which two copies of a stem-loop structure (identified as increasing inhibition due to incorporation into RISC and other complexes) were present, also driven by the same SM22a promoter.

TuD RNA comprises (5′-3′):
(SEQ ID NO: 48)
GACGGCGCTAGGATCATC-AAC-AGGGATTCCTATCTTTTAAACTGG
ACCAA-GTATTCTGGTCAAGAATAC-AAC-GAGCTACAGTATCTGC
TTCATCTGA-CAAGATGATCCTAGCGCCGTC-TTTTTTT

### Example 9. AAV Expression of 4-miRNA Cassette Induced Endothelial Markers in Vascular Smooth Muscle Cells

The following materials and methods were used in this Example 9.

### Cell Culture

Human Coronary Artery Smooth Muscle Cells (CASMCs, Lonza) and MOVAS cells (ATCC) were cultured in smooth muscle cell growth media (SMGM, Lonza) prior to use,

### Transdifferentiation Protocol

CASMCs were seeded into 6-well plates 24 hours prior to transfection at 40-60% confluency. AAV-pSM22-GFP control and AAV-pSM22-miRNA cassette (containing miR-146a+miR181b2 and decoys miR145+miR143)-CFP were purified to 2.0×10¹³ GC/mL in PBS 5% glycerol stock (Vector Biolabs). CASMCs or MOVAS cells were transduced with AAVs at 10⁴ MOI. After 48 hours, SMGM was aspirated and EGM-2 with 8-Br-cAMP (2 μL/2 mL, 0.1 mM stock solution, Sigma-Aldrich) and SB 431542 (2 μL/mL, 10 μstock solution, Stem Cell) was used. Cells were expanded for 7 days prior to fluorescence activated cell sorting (FACS) for GFP. Sorted GFP+ cells were expanded for analysis and experimental use.

### RT-qPCR

Total RNA was extracted with QIAzol or TRIzol reagent and miRNeasy micro kit (Qiagen) per manufacturer protocol. PNA concentration and quality were assessed using Qubit (ThermoFisher) and Bioanalyzer (Agilent), Isolated RNA was reverse transcribed using a High-Capacity cDNA Reverse Transcription kit (Applied Biosystems) or miRCURY LNA miRNA RT kit (Qiagen) according to manufacturer specifications. Real-time quantitative PCR (RT-qPCR) was conducted on the AriaMx RT-qPCR system (Agilent). Specific primers include ACTA2, calponin, MYH11, vimentin, VE-Cadherin, CD31, KDR, and GAPDH were purchased (Life Technologies). All expression values were normalized to GAPDH and represent 2^{ΔCt}. The primers used are as shown above in Table A.

### Results

To demonstrate the ability of the AAV construct to transduce vascular smooth muscle cells, AAV pSM22a-GFP control was added to human coronary artery smooth cells (CASMCs) or the mouse SMC cell line MOVAS, Immunofluorescence imaging for CFP showed successful transduction of both CASMCs and MOVAS (FIG. 15).

In addition, AAV pSM22a-GFP control or AAV pSM22a-miRNA cassette-GFP was added at 10⁴ MOI to the mouse SMC cell line MOVAS or human coronary artery smooth cells (CASMCs). Cells were FACS sorted for GFP after 7 days and subjected to RTqPCR for miR-143, miR-145, miR-146a, and miR-181b. The results (see (FIGS. 16A-B) showed that AAV pSM22a-miRNA cassette-GFP successfully inhibited the expression of miR-143 and miR-145 and overexpressed miR-146a and miR-181b in both cell lines.

AAV pSM22a-GFP control or AAV pSM22a-miRNA cassette-GFP was added at 10⁴ MOI to human coronary artery smooth cells (CASMCs) or the mouse SMC cell line MOVAS. Cells were FACS soiled for GFP after 7 days and subjected to RTqPCR for endothelial markers CD31, KDR and VE-cadherin and vascular smooth muscle cell markers ACTA2, vimentin, TGLN/CNN1, or Myh11 as indicated. The results (see FIGS. 17A-D) showed that the AAV pSM22a-miRNA cassette-GFP successfully increased endothelial markers and repressed vascular smooth muscle cell markers.

### REFERENCES

1. C. Michiels, Endothelial cell functions. Journal of Cellular Physiology 196, 430-443 (2003).
2. M. A. Gimbrone, G. García-Cardeña, Endothelial Cell Dysfunction and the Pathobiology of Atherosclerosis. Circulation Research 118. 620-636 (2016).
3. P. Libby, P. M. Ridker, G. K. Hansson, Inflammation in Atherosclerosis. Journal of the American College of Cardiology 54, 2129-2138 (2009).
4. J. Thyberg, Re-endothelialization Via Bone Marrow-Derived Progenitor Cells. Arteriosclerosis, Thrombosis, and Vascular Biology 22, 1509-1511 (2002).
5. T. Yamamoto et al., Therapeutic Reendothelialization by Induced Pluripotent Stein Cells After Vascular Injury-Brief Report. Arteriosclerosis, Thrombosis, and Vascular Biology 33, 2218-2221 (2013).
6. N. Saved et al., Transdifferentiation of Human Fibroblasts to Endothelial Cells. Circulation 131, 300-309 (2015).
7. C. Fernández-Hernando, Y, Suarez, MicroRNAs in endothelial cell homeostasis and vascular disease. Current Opinion in Hematology 25, 227-236 (2018).
8. Y. SuáRez, C. FernáNdez-Hernando, J. S. Pober, W. C. Sessa, Dicer Dependent MicroRNAs Regulate Gene Expression and Functions in Human Endothelial Cells. Circulation Research 100, 1164-1173 (2007).
9. D. P. Bartel, MicroRNAs: Target Recognition and Regulatory Functions. Cell 136, 215-233 (2009).
10. P. Landgraf et al., A Mammalian microRNA Expression Atlas Based on Small RNA Library Sequencing. Cell 129, 1401-1414 (2007).
11. M. W. Feinberg, K. J. Moore, MicroRNA Regulation of Atherosclerosis. Circulation Research 118, 703-720 (2016).
12. H. Ling, M, Fabbri, G. A. Calm, MicroRNAs and other non-coding RNAs as targets for anticancer drug development. Nature Reviews Drug Discovery
12. 847-865 (2013).
13. Z. Song, G. Li, Role of Specific MicroRNAs in Regulation of Vascular Smooth Muscle Cell Differentiation and the Response to Injury. Journal of Cardiovascular Translational Research 3, 246-250 (2010).
14. X. Liu et al., A Necessary Role of miR-221 and miR-222 in Vascular Smooth Muscle Cell Proliferation and Neointimal Hyperplasia. Circulation Research 104, 476-487 (2009).
15. M. Climent et al., TGFβ Triggers miR-143/145 Transfer From Smooth Muscle Cells to Endothelial Cells, Thereby Modulating Vessel Stabilization. Circulation Research 116, 1753-1764 (2015).
16. E. Hergenreider et al., Atheroprotective communication between endothelial cells and smooth muscle cells through miRNAs. Nature Cell Biology 14, 249-256 (2012).
17. J. Kim, MicroRNAs as critical regulators of the endothelial to mesenchymal transition in vascular biology. BMB Reports 51, 65-72 (2018).
18. Z. Gu, L. Gu, R. Eils, M. Schlesner, B. Brors, circlize implements and enhances circular visualization in R, Bioinformatics 30, 2811-2812 (2014).
19. N. Robbins, A. Thompson, A. Mann, A. L. Blomkalns, Isolation and Excision of Murine Aorta; A Versatile Technique in the Study of Cardiovascular Disease. Journal of Visualized Experiments, (2014).
20. K. R, Cordes et al., miR-145 and miR-143 regulate smooth muscle cell fate and plasticity. Nature 460, 705-710 (2009).
21. J. Li et al., miR-146a, an IL-1beta responsive miRNA, induces vascular endothelial growth factor and chondrocyte apoptosis by targeting Smad4. Arthritis Res Ther 14, R75 (2012).
22. X. Sun, A, Sit, M. W. Feinberg, Role of miR-181 family in regulating vascular inflammation and immunity. Trends in Cardiovascular Medicine 24, 105-112 (2014).
23. M, Ginsberg et al., Efficient direct reprogramming of mature amniotic cells into endothelial cells by ETS factors and TGFbeta suppression. Cell 151, 559-575 (2012).
24. T. Watabe et al., TGF-beta receptor kinase inhibitor enhances growth and integrity of embryonic stein cell-derived endothelial cells. J Cell Biol 163, 1303-1311 (2003).
25. N. Sayed et al., Transdifferentiation of human fibroblasts to endothelial cells: role of innate immunity. Circulation 131, 300-309 (2015).
26. X. Hong et al., Transdifferentiated Human Vascular Smooth Muscle Cells are a New Potential Cell Source for Endothelial Regeneration. Scientific Reports 7, (2017).
27. D. Wang, A. G. Atanasov, The microRNAs Regulating; Vascular Smooth Muscle Cell Proliferation: A Minireview. International Journal of Molecular Sciences 20, 324 (2019).
28. T. Boettger et al., Acquisition of the contractile phenotype by murine arterial smooth muscle cells depends on the Mir143/145 gene cluster. J Clin Invest 119, 2634-2647 (2009).
29. X. Sun et al., MicroRNA-181b regulates NF-κB-mediated vascular inflammation. Journal of Clinical Investigation, (2012).
30. X. Wang et al., Increased expression of microRNA-146a decreases myocardial ischaemia/reperfusion injury. Cardiovascular Research 97, 432-442 (2013).
31. A. Y. Rangrez, Z. A. Massy, V. Metzinger-Le Meuth, L. Metzinger, miR-143 and miR-145: molecular keys to switch the phenotype of vascular smooth muscle cells. Circ Cardiovasc Genet 4, 197-205 (2011).
32. L. Deng et al., MicroRNA-143 Activation Regulates Smooth Muscle and Endothelial Cell Crosstalk in Pulmonary Arterial Hypertension. Circulation research 117, 870-883 (2015).
33. H. Ni et al., Smooth Muscle Cell-Enriched Long Noncoding RNA Regulates Cell Plasticity and Atherosclerosis by Interacting With Serum Response Factor. Arterioscler Thromb Vase Biol, ATVBAHA120315911 (2021).
34. M. Xin et al., MicroRNAs miR-143 and miR-145 modulate cytoskeletal dynamics and responsiveness of smooth muscle cells to injury. Genes & Development 23, 2166-2178 (2009).
35. L. Elia et al., The knockout of miR-143 and -145 alters smooth muscle cell maintenance and vascular homeostasis in mice: correlates with human disease. Cell Death & Differentiation 16, 1590-1598 (2009).
36. S. Yang et al., miR-145 regulates myofibroblast differentiation and lung; fibrosis. The FASEB Journal 27, 2382-2391 (2013).
37. L. Deng et al., MicroRNA-143 Activation Regulates Smooth Muscle and Endothelial Cell Crosstalk in Pulmonary Arterial Hypertension. Circulation Research 117, 870-883 (2015).
38. M. Climent et al., TGFbeta Triggers miR-143/145 Transfer From Smooth Muscle Cells to Endothelial Cells, Thereby Modulating Vessel Stabilization. Cire Res 116, 1753-1764 (2015).
39. M. Fontaine et al., Extracellular Vesicles Mediate Communication between Endothelial and Vascular Smooth Muscle Cells. International Journal of Molecular Sciences 23, 331 (2021).
40. H. S. Cheng et al., Micro RNA-146 represses endothelial activation by inhibiting pro-inflammatory pathways. EMBO Molecular Medicine 5, 1017-1034 (2013).
41. M. R. Rippo et al., MitomiRs in human inflamm-aging: a hypothesis involving miR-181a, miR-34a and miR-146a. Exp Gerontol 56, 154-163 (2014).
42. L. Guo et al, MicroRNAs, TGF-β signaling, and the inflammatory microenvironment in cancer. Tumor Biology 37, 115-125 (2016).
43. X. Sun et al., MicroRNA-181b Improves Glucose Homeostasis and Insulin Sensitivity by Regulating Endothelial Function in White Adipose Tissue, Circulation Research 118, 810-821 (2016).
44. D. Hori et al., miR-181b regulates vascular stiffness age dependently in part by regulating TGF-β signaling. PLOS ONE 12 e0174108 (2017).
45. X. Sun et al., Systemic delivery of microRNA-181b inhibits nuclear factor-kappaB activation, vascular inflammation, and atherosclerosis in apolipoprotein E-deficient mice. Circ Res 114, 32-40 (2014).
46. J. Lin et al., MicroRNA-181b inhibits thrombin-mediated endothelial activation and arterial thrombosis by targeting caspase recruitment domain family member 10. FASEB J 30, 3216-3226 (2016)
47. R. L. Judson, T. S. Greve, R. J. Parchem, R. Blelloch, MicroRNA-based discovery of barriers to dedifferentiation of fibroblasts to pluripotent stem cells. Nature Structural & Molecular Biology 20, 1227-1235 (2013).
48. X. Sun, N. Belkin, M. W. Feinberg, Endothelial microRNAs and atherosclerosis. Curr Atheroscler Rep 15, 372 (2013).
49. N. M. Kane et al., Role of MicroRNAs 99b, 181a, and 181b in the Differentiation of Human Embryonic Stem Cells to Vascular Endothelial Cells. STEM CELLS 30, 643-654 (2012).
50. Y. Su et al., MicroRNA-181a-5p and microRNA-181a-3p cooperatively restrict vascular inflammation and atherosclerosis. Cell Death & Disease 10, (2019).
51. E. Giacomelli et al., Human-iPSC-Derived Cardiac Stromal Cells Enhance Maturation in 31D Cardiac Microtissues and Reveal Non-cardiomyocyte Contributions to Heart Disease, Cell Stem Cell 26, 862-879.e811 (2020).
52. T. M. Jayawardena et al., MicroRNA-Mediated In Vitro and In Vivo Direct Reprogramming of Cardiac Fibroblasts to Cardiomyocytes. Circulation Research 110, 1465-1473 (2012).
53. F. Cipollone et al, A Unique MicroRNA Signature Associated With Plaque Instability in Humans. Stroke 42, 2556-2563 (2011).
54. Y, J, Nam et al, Reprogramming of human fibroblasts toward a cardiac fate. Proceedings of the National Academy of Sciences 110, 5588-5593 (2013).
55. K. Tréguer, E.-M. Heinrich, K. Ohtani, A. Bonauer, S. Dimmeler, Role of the MicroRNA-17-92 Cluster in the Endothelial Differentiation of Stem Cells. Journal of Vascular Research 49, 447-460 (2012),
56. Z. Luo et al., MicroRNA-200C and -150 play an important role in endothelial cell differentiation and vasculogenesis by targeting transcription repressor ZEBI. STEM CELLS 31, 1749-1762 (2013),
57. B. Feng et al., miR-200b Mediates Endothelial-to-Mesenchymal Transition in Diabetic Cardiomyopathy. Diabetes 65, 768-779 (2016).
58. Y. Wang et al., Tumor-Derived EV-Encapsulated miR-181b-5p Induces Angiogenesis to Foster Tumorigenesis and Metastasis of ESCC. Mol Ther Nucleic Acids 20, 421-437 (2020).
59. N. Nagai et al., Downregulation of ERG and FLI1 expression in endothelial cells triggers endothelial-to-mesenchymal transition. PLOS Genetics 14, e1007826 (2018).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for generating a population of inducible endothelial cells (iECs), the method comprising:
(a) providing a population of cells comprising smooth muscle cells (SMCs) or fibroblasts;
(b) contacting the population of cells comprising SMCs or fibroblasts with constructs for expression of transdifferentiation factors, wherein the constructs comprise one or both of (i) one, two, or more tough decoy RNA expression sequences for inhibiting miR-143 and miR-145 that is at least 80%, 85%, 90%, 95%, or 99% identical to SEQ ID NO:48, and/or (ii) a miR-146a/miR-181b-2 miRNA mimic expression sequence that is at least 80%, 85%, 90%, 95%, or 99% identical to SEQ ID NO:43, in an amount and for a time sufficient to induce transdifferentiation of the SMCs or fibroblasts to iECs, and
(c) culturing the cells in supportive media, preferably for at least 10, 12, 14, 16, 18, or 20 days,
thereby generating a population of iECs.

2. The method of claim 1, wherein the SMC are isolated from aorta (AoSMC), coronary artery (CASMC), pulmonary artery (PASMC), umbilical artery (UASMC), bladder smooth muscle cells (HBdSMC), or derived from adipose tissue smooth muscle cells or progenitors (ASMCs), or blood derived circulating smooth muscle cell progenitors (SPCs); or wherein the fibroblasts are from skin (dermal fibroblasts), lung fibroblasts, cardiac fibroblasts, aortic fibroblasts, adipose tissue fibroblasts, or foreskin fibroblasts.

3. The method of claim 1, wherein the SMCs or fibroblasts are contacted in vitro or in vivo.

4. The method of claim 1, wherein the transdifferentiation factors are administered as two different constructs or as one combined construct.

5. The method of claim 4, wherein the constructs are administered in a composition comprising liposomes, optionally wherein the liposomes encapsulate the constructs.

6. The method of claim 1, wherein the constructs are administered in a plasmid or viral vector.

7. The method of claim 6, wherein the viral vector is a retrovirus, adenovirus, adeno-associated virus, or lentivirus.

8. The method of claim 7, wherein the viral vector is an AAV, preferably AAV2, AAV2.5, or AAV9.

9. The method of claim 1, wherein the construct further comprises a promoter for expression in smooth muscle cells, optionally SM22A promoter.

10. A composition comprising one or more expression constructs for expression of transdifferentiation factors, wherein the expression construct comprises one or both of (i) one, two, or more tough decoy RNA expression sequences for inhibiting miR-143 and miR-145 that is at least 80%, 85%, 90%, 95%, or 99% identical to SEQ ID NO:48, and/or (ii) a miR-146a/miR-181b-2 miRNA mimic expression sequence that is at least 80%, 85%, 90%, 95%, or 99% identical to SEQ ID NO:43, optionally in a carrier, optionally a pharmaceutically acceptable carrier.

11. The composition of claim 10, wherein the composition comprises liposomes, optionally wherein the liposomes encapsulate the one or more expression constructs.

12. The composition of claim 10, wherein the expression construct is a plasmid or viral vector.

13. The composition of claim 12, wherein the viral vector is a retrovirus, adenovirus, adeno-associated virus, or lentivirus.

14. The composition of claim 12, wherein the viral vector is an AAV, preferably AAV2, AAV2.5, or AAV9.

15. The composition of claim 1, wherein the construct further comprises a promoter for expression in smooth muscle cells, optionally SM22A promoter, and/or an enhancer, optionally woodchuck hepatitis virus post-transcriptional regulatory element (WPRE).

16. A method of treating a subject, the method comprising administering to the subject a therapeutically effective amount of:
(i) a population of inducible endothelial cells (iECs) generated by the method of claim 1; or
(ii) a composition comprising one or more expression constructs for expression of transdifferentiation factors, wherein the expression construct comprises one or both of (i) one, two, or more tough decoy RNA expression sequences for inhibiting miR-143 and miR-145 that is at least 80%, 85%, 90%, 95%, or 99% identical to SEQ ID NO:48, and/or (ii) a miR-146a/miR-181b-2 miRNA mimic expression sequence that is at least 80%, 85%, 90%, 95%, or 99% identical to SEQ ID NO:43, optionally in a carrier, optionally a pharmaceutically acceptable carrier.

17. The method of claim 16, wherein the subject has or is at risk of developing endothelial injury.

18. The method of claim 17, wherein the subject has or is at risk of developing endothelial injury as a result of a planned or past cardiovascular intervention.

19. The method of claim 18, wherein the cardiovascular intervention comprises angioplasty, stent placement, catheter ablation, heart valve surgery, or bypass surgery.

20. The method of claim 17, wherein the subject has a disorder associated with ischemic injury.

21. The method of claim 20, wherein the disorder associated with ischemic injury is myocardial infarction, ischemic stroke, ischemic renal injury, limb ischemia, arteriovenous (AV) fistula injury, organ transplant graft injury, or a wound healing.


SEQUENCE LISTING (48 sequences)

SEQ ID NO: 1 - RNA, 21 nt, Homo sapiens
  source 1..21: mol_type = other RNA
tgagatgaagcactgtagctc

SEQ ID NO: 2 - RNA, 23 nt, Homo sapiens
  source 1..23: mol_type = other RNA
gtccagttttcccaggaatccct

SEQ ID NO: 3 - RNA, 22 nt, Homo sapiens
  source 1..22: mol_type = other RNA
tgagaactgaattccatgggtt

SEQ ID NO: 4 - RNA, 23 nt, Homo sapiens
  source 1..23: mol_type = other RNA
aacattcattgctgtcggtgggt

SEQ ID NO: 5 - DNA, 23 bp, synthetic construct
  source 1..23: mol_type = other DNA
agggattcctgggttttctggac

SEQ ID NO: 6 - DNA, 21 bp, synthetic construct
  source 1..21: mol_type = other DNA
gagctacagtgcttcatctca

SEQ ID NO: 7 - DNA, 99 bp, synthetic construct
  source 1..99: mol_type = other DNA
ccgatgtgtatcctcagctttgagaactgaattccatgggttgtgtcagtgtcagacctctgaaattcagttcttcagctgggatatctctgtcatcgt

SEQ ID NO: 8 - DNA, 89 bp, synthetic construct
  source 1..89: mol_type = other DNA
ctgatggctgcactcaacattcattgctgtcggtgggtttgagtctgaatcaactcactgatcaatgaatgcaaactgcggaccaaaca

SEQ ID NO: 9 - DNA, 21 bp, synthetic construct
  source 1..21: mol_type = other DNA
aaaagacagctacgtgggtga

SEQ ID NO: 10 - DNA, 22 bp, synthetic construct
  source 1..22: mol_type = other DNA
gccatgttctatcgggtacttc

SEQ ID NO: 11 - DNA, 21 bp, synthetic construct
  source 1..21: mol_type = other DNA
ctgtcagccgaggttaagaac

SEQ ID NO: 12 - DNA, 21 bp, synthetic construct
  source 1..21: mol_type = other DNA
gaggccgtccatgaagttgtt

SEQ ID NO: 13 - DNA, 19 bp, synthetic construct
  source 1..19: mol_type = other DNA
cgccaagagactcgtctgg

SEQ ID NO: 14 - DNA, 21 bp, synthetic construct
  source 1..21: mol_type = other DNA
tctttcccaaccgtgaccttc

SEQ ID NO: 15 - DNA, 20 bp, synthetic construct
  source 1..20: mol_type = other DNA
gacgccatcaacaccgagtt

SEQ ID NO: 16 - DNA, 21 bp, synthetic construct
  source 1..21: mol_type = other DNA
ctttgtcgttggttagctggt

SEQ ID NO: 17 - DNA, 22 bp, synthetic construct
  source 1..22: mol_type = other DNA
ttggaaccagatgcacattgat

SEQ ID NO: 18 - DNA, 20 bp, synthetic construct
  source 1..20: mol_type = other DNA
tcttgcgactcacgcttgac

SEQ ID NO: 19 - DNA, 22 bp, synthetic construct
  source 1..22: mol_type = other DNA
aacagtgttgacatgaagagcc

SEQ ID NO: 20 - DNA, 22 bp, synthetic construct
  source 1..22: mol_type = other DNA
tgtaaaacagcacgtcatcctt

SEQ ID NO: 21 - DNA, 21 bp, synthetic construct
  source 1..21: mol_type = other DNA
ggcccaataatcagagtggca

SEQ ID NO: 22 - DNA, 23 bp, synthetic construct
  source 1..23: mol_type = other DNA
ccagtgtcatttccgatcacttt

SEQ ID NO: 23 - DNA, 19 bp, synthetic construct
  source 1..19: mol_type = other DNA
ccgatgcagccttttcgga

SEQ ID NO: 24 - DNA, 21 bp, synthetic construct
  source 1..21: mol_type = other DNA
tccccaagatacacggagagg

SEQ ID NO: 25 - DNA, 21 bp, synthetic construct
  source 1..21: mol_type = other DNA
agagtggagcctggtcttaca

SEQ ID NO: 26 - DNA, 23 bp, synthetic construct
  source 1..23: mol_type = other DNA
cctttgctgacaataagcactgg

SEQ ID NO: 27 - DNA, 21 bp, synthetic construct
  source 1..21: mol_type = other DNA
gggaagatggtcgtgatcctt

SEQ ID NO: 28 - DNA, 21 bp, synthetic construct
  source 1..21: mol_type = other DNA
tctggggtggtctcgatttta

SEQ ID NO: 29 - DNA, 19 bp, synthetic construct
  source 1..19: mol_type = other DNA
cggctgcttcgcagaatca

SEQ ID NO: 30 - DNA, 22 bp, synthetic construct
  source 1..22: mol_type = other DNA
tcttggggatctccaatgtgag

SEQ ID NO: 31 - DNA, 20 bp, synthetic construct
  source 1..20: mol_type = other DNA
acggcgttacagtgtttctg

SEQ ID NO: 32 - DNA, 22 bp, synthetic construct
  source 1..22: mol_type = other DNA
gcacatacaaacggcctatctc

SEQ ID NO: 33 - DNA, 23 bp, synthetic construct
  source 1..23: mol_type = other DNA
gtagctctgatgagtgcaatgac

SEQ ID NO: 34 - DNA, 21 bp, synthetic construct
  source 1..21: mol_type = other DNA
cagatatggcaactcccagtg

SEQ ID NO: 35 - DNA, 21 bp, synthetic construct
  source 1..21: mol_type = other DNA
agacacgggagtgcatctact

SEQ ID NO: 36 - DNA, 22 bp, synthetic construct
  source 1..22: mol_type = other DNA
gcctatcgtagcagttgaagtc

SEQ ID NO: 37 - DNA, 21 bp, synthetic construct
  source 1..21: mol_type = other DNA
ggagcgagatccctccaaaat

SEQ ID NO: 38 - DNA, 23 bp, synthetic construct
  source 1..23: mol_type = other DNA
ggctgttgtcatacttctcatgg

SEQ ID NO: 39 - RNA, 99 nt, synthetic construct
  source 1..99: mol_type = other RNA
ccgatgtgtatcctcagctttgagaactgaattccatgggttgtgtcagtgtcagacctctgaaattcagttcttcagctgggatatctctgtcatcgt

SEQ ID NO: 40 - RNA, 89 nt, synthetic construct
  source 1..89: mol_type = other RNA
ctgatggctgcactcaacattcattgctgtcggtgggtttgagtctgaatcaactcactgatcaatgaatgcaaactgcggaccaaaca

SEQ ID NO: 41 - DNA, 599 bp, synthetic construct
  source 1..599: mol_type = other DNA
tttagtagagacaaattctccatgttgcccaggctagtcctgaactcctgggctcaagagatccacccacatcagccttccagactgctggcctggtctcctccagatgtttataactcatgagtgccaggactagacctggtactaggaagcagctgcattggatttaccaggcttttcactcttgtattttacagggctgggacaggcctggactgcaaggaggggtctttgcaccatctctgaaaagccgatgtgtatcctcagctttgagaactgaattccatgggttgtgtcagtgtcagacctctgaaattcagttcttcagctgggatatctctgtcatcgtgggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctactcgtttacatttattgattact

SEQ ID NO: 42 - DNA, 588 bp, synthetic construct
  source 1..588: mol_type = other DNA
gcctgtgtggcgcagtgctacctgtgaggttctccaagcactccttccttctctgaaccacagcttcctcatctgcaataacctcccagctccaatgtcaatgttatggacacctgtgtgggccctcaatcatgcagatggctggttactaagggagaagccagacacacagacttcaaagaactgagatggaaaagaagagccaggagtcagccagggagggcaaaggcaaccccaccaactgaaaacactgatggctgcactcaacattcattgctgtcggtgggtttgagtctgaatcaactcactgatcaatgaatgcaaactgcggaccaaacacccaaaggaagcaaaagcatgtgcagagagtcgcctgagtgcagcagctactgctgtgagtgggagctgctactatttccaaaacactagctaaagcttttcttgttctccttggttctaacatggagtaagttctctaaattcctagtacttttctcctttttcaatagtctagatttaaggaaatgatattttataggaagaattaaatacatatctggatatcacatcacattttaaaaatcagta

SEQ ID NO: 43 - DNA, 1141 bp, synthetic construct
  source 1..1141: mol_type = other DNA
tttagtagagacaaattctccatgttgcccaggctagtcctgaactcctgggctcaagagatccacccacatcagccttccagactgctggcctggtctcctccagatgtttataactcatgagtgccaggactagacctggtactaggaagcagctgcattggatttaccaggcttttcactcttgtattttacagggctgggacaggcctggactgcaaggaggggtctttgcaccatctctgaaaagccgatgtgtatcctcagctttgagaactgaattccatgggttgtgtcagtgtcagacctctgaaattcagttcttcagctgggatatctctgtcatcgtgggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctatgaggttctccaagcactccttccttctctgaaccacagcttcctcatctgcaataacctcccagctccaatgtcaatgttatggacacctgtgtgggccctcaatcatgcagatggctggttactaagggagaagccagacacacagacttcaaagaactgagatggaaaagaagagccaggagtcagccagggagggcaaaggcaaccccaccaactgaaaacactgatggctgcactcaacattcattgctgtcggtgggtttgagtctgaatcaactcactgatcaatgaatgcaaactgcggaccaaacaacccaaaggaagcaaaagcatgtgcagagagtcgcctgagtgcagcagctactgctgtgagtgggagctgctactatttccaaaacactagctaaagcttttcttgttctccttggttctaacatggagtaagttctctaaattcctagtacttttctcctttttcaatagtctagatttaaggaaatgatattttataggaagaattaaatacatatctggatatcacatcacattttaaaaatcagta

SEQ ID NO: 44 - DNA, 22 bp, synthetic construct
  source 1..22: mol_type = other DNA
agggattccttttaaactggac

SEQ ID NO: 45 - DNA, 21 bp, synthetic construct
  source 1..21: mol_type = other DNA
gagctacagtgcttcatctga

SEQ ID NO: 46 - DNA, 152 bp, synthetic construct
  source 1..152: mol_type = other DNA
agggattccttttaaactggacaattagggattccttttaaactggacaattagggattccttttaaactggacaattagggattccttttaaactggacaattagggattccttttaaactggacaattagggattccttttaaactggac

SEQ ID NO: 47 - DNA, 146 bp, synthetic construct
  source 1..146: mol_type = other DNA
gagctacagtgcttcatctgaaattgagctacagtgcttcatctgaaattgagctacagtgcttcatctgaaattgagctacagtgcttcatctgaaattgagctacagtgcttcatctgaaattgagctacagtgcttcatctga

SEQ ID NO: 48 - DNA, 125 bp, synthetic construct
  source 1..125: mol_type = other DNA
gacggcgctaggatcatcaacagggattcctatcttttaaactggaccaagtattctggtcaagaatacaacgagctacagtatctgcttcatctgacaagatgatcctagcgccgtcttttttt